(19) United States
(12) Patent Application Publication
(10) Pub. No.: US 2022/0399125 A1
(43) Pub. Date: Dec. 15, 2022

(54) COMPUTER SYSTEMS AND METHODS FOR MACHINE-LEARNING BASED SEVERITY MODELING FOR ONCOLOGY BASED ON INCONSISTENT CANCER STAGE DATA RECORDS

(71) Applicant: Optum, Inc., Minnetonka, MN (US)

(72) Inventors: Christine M. John, Hermitage, TN (US); Lorre Ann Ochs, Eden Prairie, MN (US); Lynn Anne Richards, Brooklyn Park, MN (US); Fang Guo, Apex, NC (US); Liping Fan, Basking Ridge, NJ (US); Julia Vaynerman, Eagan, MN (US)

(21) Appl. No.: 17/344,466

(22) Filed: Jun. 10, 2021

(51) Int. Cl. G16H 50/50; G06F 30/27; G06N 20/00; G06N 5/04; G16H 10/60; G16H 50/20; G16H 50/70; A61B 5/00

(52) U.S. Cl. CPC G16H 50/50; G06F 30/27; G06N 20/00; G06N 5/04; G16H 10/60; G16H 50/20; G16H 50/70; A61B 5/4842

(57) ABSTRACT

To automatically model oncology treatment severity levels for a particular patient, a plurality of independently generated and potentially inconsistent observation data records are received for a patient. A condition-specific plurality of observation data records each having a common cancer type identifier are selected, and those condition-specific observation records are filtered to potentially eliminate one or more observation data records failing to satisfy one or more preliminary filter criteria. A specific pre-processing process for the remaining observation data records is executed to generate one or more output data records having at least one shared identifier, and the output data records are provided as model input to a machine-learning based severity model to generate a severity score for the patient.

### BACKGROUND

Oncological diagnosis and treatment of many cancer types often results in a large number of data records for each patient. Those data records generally encompass clinical data records that may encompass notes of a medical care provider, lab data records indicating the results of lab tests performed on the patient, and/or the like. In addition, the data records relating to oncological diagnosis and treatment may further encompass clinical data records including indications of one or more diagnoses associated with the patient, and/or the like. These data records are generated independently from one another, which can periodically result in inconsistent data describing aspects of a patient's cancer and/or treatment. These inconsistencies within data for a particular patient may inhibit use of automated computer-based models that rely on consistent data to produce accurate model outputs.

Accordingly, a need exists for systems and methods that enable computer-implemented models to intake and reconcile potentially inconsistent data sets for use with computer-implemented data models.

### BRIEF SUMMARY

Various embodiments comprise pre-processing systems and methods for oncological data indicative of a cancer stage for a patient that are configured to generate consistent output data sets that may be utilized as input for further analytics including, but not limited to, computer-based modeling, such as machine-learning based models. The pre-processing systems and methods comprise a plurality of cancer type-specific stage pre-processing rules to be applied in a sequential manner. Upon generating a model input data set via the pre-processing systems and methods, various embodiments provide the model input data set to one or more machine-learning based models configured for generating severity data associated with the patient's cancer.

Certain embodiments are directed to a computer-implemented method for automatically modeling severity attributes of a cancer treatment utilizing a plurality of independently generated observation data records for a patient. In certain embodiments, the method comprises: receiving a plurality of independently generated observation data records each comprising structured observation data for a patient; identifying a condition-specific plurality of observation data records selected from the plurality of independently generated observation data records, wherein the condition-specific plurality of observation data records are embodied as observation data records all comprising a common cancer type identifier; filtering the plurality of condition-specific observation data records to eliminate one or more data records failing to satisfy one or more preliminary filter criteria; based at least in part on the common cancer type identifier, initiating a pre-processing process for the condition-specific observation data records, wherein the pre-processing process sequentially executes a plurality of subprocesses to exclude one or more data records and to generate one or more output data records for the condition-specific observation data records having at least one shared identifier; providing the one or more output data records to a selected machine-learning based model selected from a plurality of machine-learning based models based at least in part on one or more identifiers present within at least one of the one or more output data records; and generating, via the selected machine-learning based model, a severity score indicative of one or more severity attributes for the patient.

In certain embodiments, initiating a pre-processing process further comprises: selecting a pre-processing process for the condition-specific observation data records from a plurality of available pre-processing processes comprising: a first available pre-processing process applicable to one or more first cancer type identifiers, wherein the first available pre-processing process is configured to output a model input data set comprising one or more data records comprising a relevant cancer stage identifier selected from a first plurality of cancer stage identifiers applicable to the one or more first cancer type identifiers; a second available pre-processing process applicable to one or more second cancer type identifiers, wherein the second available pre-processing process is configured to output a model input data set comprising one or more data records comprising a relevant cancer stage identifier selected from a second plurality of cancer stage identifiers applicable to the one or more second cancer type identifiers; and a third available pre-processing process applicable to one or more third cancer type identifiers, wherein the third available pre-processing process is configured to output a model input data set comprising one or more data records comprising a relevant cancer stage identifier selected from a third plurality of cancer stage identifiers applicable to the one or more third cancer type identifiers.

In various embodiments, the one or more first cancer type identifiers comprise a Small-Cell Lung Cancer (SCLC) identifier, and the first available pre-processing process is configured to output a model input data set comprising one or more data records comprising a relevant cancer stage identifier selected from a limited stage identifier or an extensive stage identifier; the one or more second cancer type identifiers comprise one or more of: (a) a breast cancer identifier, (b) a colon cancer identifier, (c) a rectal cancer identifier, or (d) a Non-Small-Cell Lung Cancer (NSCLC) identifier; and the second available pre-processing process is configured to output a model input data set comprising one or more data records comprising a relevant cancer stage identifier selected from: a Stage 0 identifier, a Stage I identifier, a Stage II identifier, a Stage III identifier, or a Stage IV identifier; and the one or more third cancer type identifiers comprise a prostate cancer identifier, and the third available pre-processing process is configured to output a model input data set comprising one or more data records comprising a relevant cancer stage identifier selected from: a Stage I identifier, a Stage II identifier, a Stage III identifier, a Stage IV identifier, a Stage IV(M0) identifier, or a Stage IV(M1) identifier.

In certain embodiments, the preliminary filter criteria comprise one or more of: a date-based filter criterion for selecting independently generated observation data records for further analysis as generated within a defined date range; a data source filter criterion for selecting independently generated observation data records for further analysis as generated by one or more defined data sources; or a data content filter criterion for selecting independently generated observation data records for further analysis as containing an identifier selected from a plurality of available identifiers eligible for further analysis. In various embodiments, the machine learning based model is a linear regression model. According to certain embodiments, the sequentially executed plurality of subprocesses are configured to: exclude one or more observation data records identified as failing to satisfy a rule of a subprocess for an intra-date conflict between cancer stage identifiers within observation data records having a common date; and exclude one or more observation data records identified as failing to satisfy a rule of a subprocess for an inter-date conflict between cancer stage identifiers within observation data records having different dates.

In various embodiments, at least one of the sequentially executed plurality of subprocesses is configured to retrieve one or more claims data records comprising diagnostic data to identify at least one observation data record to retain within the model input data set. According to certain embodiments, the at least one of the sequentially executed plurality of subprocesses is further configured to generate a derived data element within an observation data record included within the model input data set based at least in part on the claims data records.

Various embodiments are directed to a system comprising one or more memory storage areas and one or more processors for automatically modeling severity attributes of a cancer treatment utilizing a plurality of independently generated observation data records for a patient, the one or more processors are collectively configured to: receive a plurality of independently generated observation data records each comprising structured observation data for a patient; identify a condition-specific plurality of observation data records selected from the plurality of independently generated observation data records, wherein the condition-specific plurality of observation data records are embodied as observation data records all comprising a common cancer type identifier; filter the plurality of condition-specific observation data records to eliminate one or more condition-specific observation data records failing to satisfy one or more preliminary filter criteria; based at least in part on the common cancer type identifier, initiate a pre-processing process for the condition-specific observation data records, wherein the pre-processing process sequentially executes a plurality of subprocesses to exclude one or more condition-specific observation data records and to generate one or more output data records for the condition-specific observation data records having at least one shared identifier; providing the one or more output data records to a selected machine-learning based model selected from a plurality of machine-learning based models based at least in part on one or more identifiers present within at least one of the one or more output data records; and generating, via the selected machine-learning based model, a severity score indicative of one or more severity attributes for the patient.

In various embodiments, initiating a pre-processing process further comprises: selecting a pre-processing process for the condition-specific observation data records from a plurality of available pre-processing processes comprising: a first available pre-processing process applicable to one or more first cancer type identifiers, wherein the first available pre-processing process is configured to output a model input data set comprising one or more data records comprising a relevant cancer stage identifier selected from a first plurality of cancer stage identifiers applicable to the one or more first cancer type identifiers; a second available pre-processing process applicable to one or more second cancer type identifiers, wherein the second available pre-processing process is configured to output a model input data set comprising one or more data records comprising a relevant cancer stage identifier selected from a second plurality of cancer stage identifiers applicable to the one or more second cancer type identifiers; and a third available pre-processing process applicable to one or more third cancer type identifiers, wherein the third available pre-processing process is configured to output a model input data set comprising one or more data records comprising a relevant cancer stage identifier selected from a third plurality of cancer stage identifiers applicable to the one or more third cancer type identifiers.

In various embodiments, the one or more first cancer type identifiers comprise a Small-Cell Lung Cancer (SCLC) identifier, and the first available pre-processing process is configured to output a model input data set comprising one or more data records comprising a relevant cancer stage identifier selected from a limited stage identifier or an extensive stage identifier; the one or more second cancer type identifiers comprise one or more of: (a) a breast cancer identifier, (b) a colon cancer identifier, (c) a rectal cancer identifier, or (d) a Non-Small-Cell Lung Cancer (NSCLC) identifier; and the second available pre-processing process is configured to output a model input data set comprising one or more data records comprising a relevant cancer stage identifier selected from: a Stage 0 identifier, a Stage I identifier, a Stage II identifier, a Stage III identifier, or a Stage IV identifier; and the one or more third cancer type identifiers comprise a prostate cancer identifier, and the third available pre-processing process is configured to output a model input data set comprising one or more data records comprising a relevant cancer stage identifier selected from: a Stage I identifier, a Stage II identifier, a Stage III identifier, a Stage IV identifier, a Stage IV(M0) identifier, or a Stage IV(M1) identifier.

In certain embodiments, the preliminary filter criteria comprise one or more of: a date-based filter criterion for selecting independently generated observation data records for further analysis as generated within a defined date range; a data source filter criterion for selecting independently generated observation data records for further analysis as generated by one or more defined data sources; or a data content filter criterion for selecting independently generated observation data records for further analysis as containing an identifier selected from a plurality of available identifiers eligible for further analysis.

In various embodiments, the machine learning based model is a linear regression model. Moreover, in certain embodiments the sequentially executed plurality of subprocesses are configured to: exclude one or more observation data records identified as failing to satisfy a rule of a subprocess for an intra-date conflict between cancer stage identifiers within observation data records having a common date; and exclude one or more observation data records identified as failing to satisfy a rule of a subprocess for an intra-date conflict between cancer stage identifiers within observation data records having different dates. In various embodiments, at least one of the sequentially executed plurality of subprocesses is configured to retrieve one or more claims data records comprising diagnostic data to identify at least one observation data record to retain within the model input data set. In various embodiments, the at least one of the sequentially executed plurality of subprocesses is further configured to generate a derived data element within an observation data record included within the model input data set based at least in part on the claims data records.

Various embodiments are directed to a computer program product for automatically modeling severity attributes of a cancer treatment utilizing a plurality of independently generated observation data records for a patient, the computer program product comprising at least one non-transitory computer-readable storage medium having computer-readable program code portions stored therein, the computer-readable program code portions configured to: receive a plurality of independently generated observation data records each comprising structured observation data for a patient; identify a condition-specific plurality of observation data records selected from the plurality of independently generated observation data records, wherein the condition-specific plurality of observation data records are embodied as observation data records all comprising a common cancer type identifier; filter the plurality of condition-specific observation data records to eliminate one or more condition-specific observation data records failing to satisfy one or more preliminary filter criteria; based at least in part on the common cancer type identifier, initiate a pre-processing process for the condition-specific observation data records, wherein the pre-processing process sequentially executes a plurality of subprocesses to exclude one or more condition-specific observation data records and to generate one or more output data records for the condition-specific observation data records having at least one shared identifier; providing the one or more output data records to a selected machine-learning based model selected from a plurality of machine-learning based models based at least in part on one or more identifiers present within at least one of the one or more output data records; and generating, via the selected machine-learning based model, a severity score indicative of one or more severity attributes for the patient.

In various embodiments, initiating a pre-processing process further comprises: selecting a pre-processing process for the condition-specific observation data records from a plurality of available pre-processing processes comprising: a first available pre-processing process applicable to one or more first cancer type identifiers, wherein the first available pre-processing process is configured to output a model input data set comprising one or more data records comprising a relevant cancer stage identifier selected from a first plurality of cancer stage identifiers applicable to the one or more first cancer type identifiers; a second available pre-processing process applicable to one or more second cancer type identifiers, wherein the second available pre-processing process is configured to output a model input data set comprising one or more data records comprising a relevant cancer stage identifier selected from a second plurality of cancer stage identifiers applicable to the one or more second cancer type identifiers; and a third available pre-processing process applicable to one or more third cancer type identifiers, wherein the third available pre-processing process is configured to output a model input data set comprising one or more data records comprising a relevant cancer stage identifier selected from a third plurality of cancer stage identifiers applicable to the one or more third cancer type identifiers.

In various embodiments, the one or more first cancer type identifiers comprise a Small-Cell Lung Cancer (SCLC) identifier, and the first available pre-processing process is configured to output a model input data set comprising one or more data records comprising a relevant cancer stage identifier selected from a limited stage identifier or an extensive stage identifier; the one or more second cancer type identifiers comprise one or more of: (a) a breast cancer identifier, (b) a colon cancer identifier, (c) a rectal cancer identifier, or (d) a Non-Small-Cell Lung Cancer (NSCLC) identifier; and the second available pre-processing process is configured to output a model input data set comprising one or more data records comprising a relevant cancer stage identifier selected from: a Stage 0 identifier, a Stage I identifier, a Stage II identifier, a Stage III identifier, or a Stage IV identifier; and the one or more third cancer type identifiers comprise a prostate cancer identifier, and the third available pre-processing process is configured to output a model input data set comprising one or more data records comprising a relevant cancer stage identifier selected from: a Stage I identifier, a Stage II identifier, a Stage III identifier, a Stage IV identifier, a Stage IV(M0) identifier, or a Stage IV(M1) identifier.

In certain embodiments, the preliminary filter criteria comprise one or more of: a date-based filter criterion for selecting independently generated observation data records for further analysis as generated within a defined date range; a data source filter criterion for selecting independently generated observation data records for further analysis as generated by one or more defined data sources; or a data content filter criterion for selecting independently generated observation data records for further analysis as containing an identifier selected from a plurality of available identifiers eligible for further analysis.

In various embodiments, the machine learning based model is a linear regression model. In various embodiments, the sequentially executed plurality of subprocesses are configured to: exclude one or more observation data records identified as failing to satisfy a rule of a subprocess for an intra-date conflict between cancer stage identifiers within observation data records having a common date; and exclude one or more observation data records identified as failing to satisfy a rule of a subprocess for an inter-date conflict between cancer stage identifiers within observation data records having different dates.

According to certain embodiments, at least one of the sequentially executed plurality of subprocesses is configured to retrieve one or more claims data records comprising diagnostic data to identify at least one observation data record to retain within the model input data set. In certain embodiments, the at least one of the sequentially executed plurality of subprocesses is further configured to generate a derived data element within an observation data record included within the model input data set based at least in part on the claims data records.

### BRIEF DESCRIPTION OF THE SEVERAL VIEWS OF THE DRAWINGS

Reference will now be made to the accompanying drawings, which are not necessarily drawn to scale, and wherein:

FIG. 1 is an exemplary overview of a system architecture that can be used to practice various embodiments;

FIG. 2 is an example schematic of a management computing entity in accordance with certain embodiments;

FIG. 3 is an example schematic of a user computing entity in accordance with certain embodiments;

FIG. 4 is a flowchart illustrating a general input process for intaking data in accordance with certain embodiments;

FIG. 5 is a flowchart illustrating an example sequence for applying cancer-specific pre-processing methodologies in accordance with certain embodiments;

FIGS. 6A-6C show a flowchart illustrating an example pre-processing methodology for pre-processing small cell lung cancer stage data in accordance with certain embodiments;

FIGS. 7A-7C show a flowchart illustrating an example pre-processing methodology for pre-processing breast cancer, colon cancer, rectal cancer, and non-small cell lung cancer stage data in accordance with certain embodiments;

FIGS. 8A-8G show a flowchart illustrating an example pre-processing methodology for pre-processing prostate cancer stage data in accordance with certain embodiments.

### DETAILED DESCRIPTION

The present disclosure more fully describes various embodiments with reference to the accompanying drawings. It should be understood that some, but not all embodiments are shown and described herein. Indeed, the embodiments may take many different forms, and accordingly this disclosure should not be construed as limited to the embodiments set forth herein. Rather, these embodiments are provided so that this disclosure will satisfy applicable legal requirements. Like numbers refer to like elements throughout.

### OVERVIEW

Observation data and/or other types of clinical data can provide highly beneficial information about a patient's cancer which cannot be gleaned from claims data, such as cancer stage and biomarker status. However, observation data results may be inconsistent, owing at least in part to the independent nature of each interaction between the patient and healthcare services (e.g., differing stage values entered in the Electronic Health Record (EHR) by different providers). These inconsistencies may impact the performance (e.g., accuracy and/or processing speed) of computer-based analytics, such as machine-learning based severity models that utilize observation data and/or other types of clinical data as a part of training data. The inconsistencies in observation data may impact the generation and/or training of machine-learning models, such that later implementation of the trained models is incapable of generating precise data outputs that can be relied upon for user decision making. As a result, there is a need for pre-processing of observation data to identify and/or reconcile any conflicts which may exist within an input data set of observation data. Accordingly, various embodiments provide tailored pre-processing methodologies for execution by a pre-processing system for intaking data (e.g., data stored within a patient's EHR, data stored within one or more claims submitted to a healthcare payer, and/or the like) generated for each of a plurality of healthcare service interactions for the patient, and for generating a data set free of conflicting data that may be automatically reviewed, such as by a machine-learning based model for generating severity attributes, such as a severity score, a predicted treatment cost, and/or the like, for a patient's cancer diagnosis.

### Technical Problem

A complete set of data generated for a patient's cancer diagnosis and treatment often includes one or more inconsistent data records, owing at least in part to the independent nature of each interaction between the patient and healthcare services (e.g., differing stage values entered in the EHR by different providers). Regardless of the source and reason of the resulting inconsistent data records, these inconsistencies create difficulties for subsequent analytics (including automated, computer-based models (e.g., machine-learning models)) to generate accurate, precise, and relevant output. Particularly for machine learning models that utilize retrospective data sets as training data for the machine-learning models, these inconsistencies between data records may result in inaccurately trained machine learning models that do not optimally identify and model aspects of a cancer diagnosis for providing severity data (e.g., estimated treatment costs) regarding the patient's cancer diagnosis.

### Technical Solution

To address the technical challenges presented by inconsistent data records existing within a data set for a patient's cancer diagnosis and treatment, embodiments as discussed herein implement a plurality of pre-processing methodologies for identifying and rectifying data inconsistencies within a data set for a particular patient, specifically cancer stage. The pre-processing methodologies are executed by a management computing entity capable of providing the resulting pre-processed data as input directly to one or more downstream analytics, such as one or more downstream machine-learning based models, such as a severity model for generating severity data (e.g., estimated treatment costs) for a patient's cancer. The pre-processing methodologies as discussed herein are cancer-type-specific, thereby enabling an accurate identification of data inconsistencies based at least in part on stage characteristics specific to a particular cancer type. These cancer-type-specific pre-processing configurations also enable accurate determinations of proper methods for rectifying those inconsistencies, such as through exclusion of certain data records, generation of additional data and/or metadata to be associated with particular data records as data tags, and/or the like. Moreover, embodiments as discussed herein ensure that the pre-processing methodologies rectify identified data inconsistencies while minimizing data loss through filtering or other data exclusion by sequential application of data pre-processing methodologies and/or subprocesses that provide an increasingly clean output data set (to be utilized as a model input data set for a downstream severity model) as pre-processing subprocesses are applied.

### III. Computer Program Products, Methods, and Computing Devices

Embodiments of the present invention may be implemented in various ways, including as computer program products that comprise articles of manufacture. Such computer program products may include one or more software components including, for example, software objects, methods, data structures, and/or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of a data structure, apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations.

Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Architecture

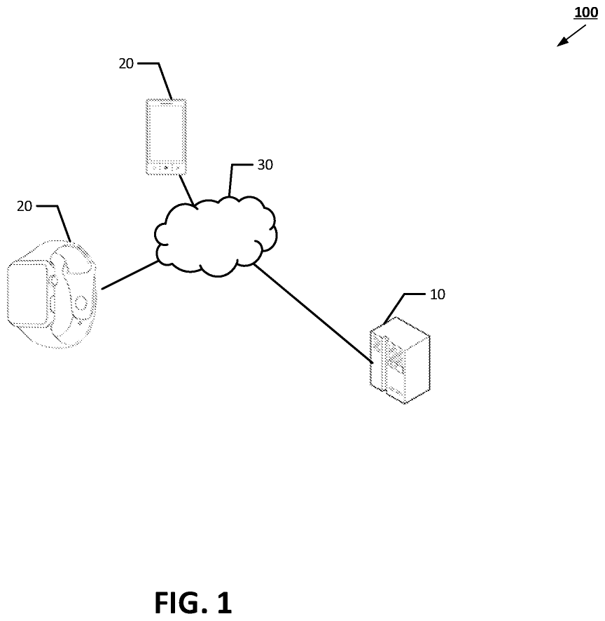

FIG. 1 provides an example system architecture 100 that can be used in conjunction with various embodiments of the present invention. As shown in FIG. 1, the system architecture 100 may comprise one or more management computing entities 10, one or more user computing entities 20, one or more networks 30, and/or the like. Each of the components of the system may be in electronic communication with, for example, one another over the same or different wireless or wired networks 30 including, for example, a wired or wireless Personal Area Network (PAN), Local Area Network (LAN), Metropolitan Area Network (MAN), Wide Area Network (WAN), and/or the like. Additionally, while FIG. 1 illustrates certain system devices as separate, standalone devices, the various embodiments are not limited to this particular architecture.

### Exemplary Management Computing Entity

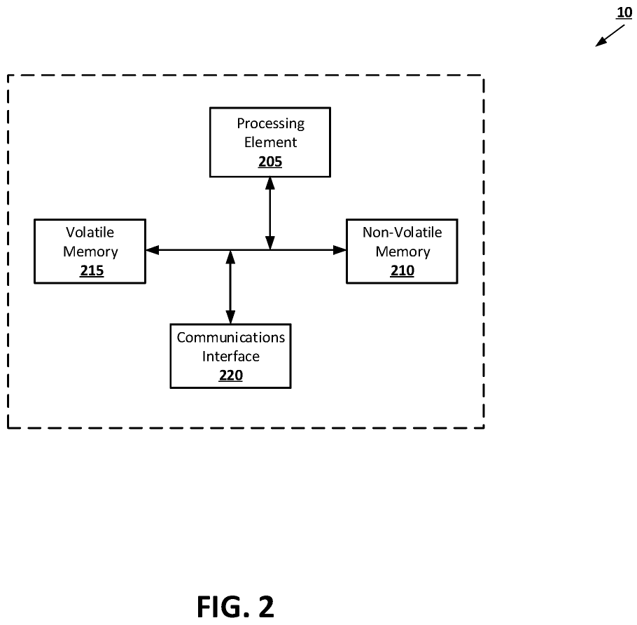

FIG. 2 provides a schematic of a management computing entity 10 according to one embodiment of the present invention. In general, the terms computing device, entity, device, system, and/or similar words used herein interchangeably may refer to, for example, one or more computers, computing devices, computing entities, desktop computers, mobile phones, tablets, phablets, notebooks, laptops, distributed systems, terminals, servers or server networks, blades, gateways, switches, processing devices, set-top boxes, relays, routers, network access points, base stations, the like, and/or any combination of devices adapted to perform the functions, operations, and/or processes described herein. Such functions, operations, and/or processes may include, for example, transmitting, receiving, operating on, processing, displaying, storing, determining, generating/creating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the management computing entity 10 may also include one or more network and/or communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the management computing entity 10 may include or be in communication with one or more processing elements 205 (also referred to as processors, processing circuitry, and/or similar terms used herein interchangeably) that communicate with other elements within the management computing entity 10 via a bus, for example. As will be understood, the processing element 205 may be embodied in a number of different ways. For example, the processing element 205 may be embodied as one or more complex programmable logic devices (CPLDs), microprocessors, multi-core processors, coprocessing devices, application-specific instruction-set processors (ASIPs), and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the management computing entity 10 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or memory media 210 as described above, such as hard disks, ROM, PROM, EPROM, EEPROM, flash memory, MMCs, SD memory cards, Memory Sticks, CBRAM, PRAM, FeRAM, RRAM, SONOS, racetrack memory, and/or the like. As will be recognized, the non-volatile storage or memory media may store databases, database instances, database management system entities, data, applications, programs, program modules, scripts, source code, object code, byte code, compiled code, interpreted code, machine code, executable instructions, and/or the like. The term database, database instance, database management system entity, and/or similar terms used herein interchangeably may refer to a structured collection of records or information/data that is stored in a computer-readable storage medium, such as via a relational database, hierarchical database, and/or network database.

In one embodiment, the management computing entity 10 may further include or be in communication with volatile media (also referred to as volatile storage, memory, memory storage, memory circuitry and/or similar terms used herein interchangeably). In one embodiment, the volatile storage or memory may also include one or more volatile storage or memory media 215 as described above, such as RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. As will be recognized, the volatile storage or memory media may be used to store at least portions of the databases, database instances, database management system entities, data, applications, programs, program modules, scripts, source code, object code, byte code, compiled code, interpreted code, machine code, executable instructions, and/or the like being executed by, for example, the processing element 205. Thus, the databases, database instances, database management system entities, data, applications, programs, program modules, scripts, source code, object code, byte code, compiled code, interpreted code, machine code, executable instructions, and/or the like may be used to control certain aspects of the operation of the management computing entity 10 with the assistance of the processing element 205 and the operating system.

As indicated, in one embodiment, the management computing entity 10 may also include one or more network and/or communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, management computing entity 10 may be configured to communicate via wireless external communication networks using any of a variety of protocols, such as general packet radio service (GPRS), Universal Mobile Telecommunications System (UMTS), Code Division Multiple Access 200 (CDMA200), CDMA200 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), IR protocols, NFC protocols, RFID protocols, IR protocols, ZigBee protocols, Z-Wave protocols, 6LoWPAN protocols, Wibree, Bluetooth protocols, wireless universal serial bus (USB) protocols, and/or any other wireless protocol. The management computing entity 10 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

As will be appreciated, one or more of the management computing entity's components may be located remotely from other management computing entity 10 components, such as in a distributed system. Furthermore, one or more of the components may be aggregated and additional components performing functions described herein may be included in the management computing entity 10. Thus, the management computing entity 10 can be adapted to accommodate a variety of needs and circumstances, such as including various components described with regard to a mobile application executing on the user computing entity 20, including various input/output interfaces.

### Exemplary User Computing Entity

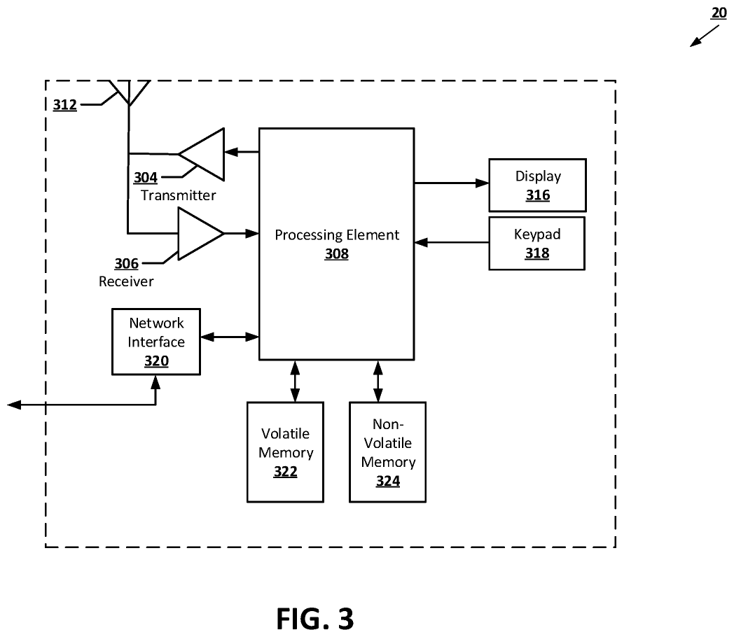

FIG. 3 provides an illustrative schematic representative of user computing entity 20 that can be used in conjunction with embodiments of the present invention. In various embodiments, the user computing entity 20 may be or comprise one or more mobile devices, wearable computing devices, and/or the like.

As shown in FIG. 3, a user computing entity 20 can include an antenna 312, a transmitter 304 (e.g., radio), a receiver 306 (e.g., radio), and a processing element 308 that provides signals to and receives signals from the transmitter 304 and receiver 306, respectively. The signals provided to and received from the transmitter 304 and the receiver 306, respectively, may include signaling information/data in accordance with an air interface standard of applicable wireless systems to communicate with various devices, such as a management computing entity 10, another user computing entity 20, and/or the like. In an example embodiment, the transmitter 304 and/or receiver 306 are configured to communicate via one or more SRC protocols. For example, the transmitter 304 and/or receiver 306 may be configured to transmit and/or receive information/data, transmissions, and/or the like of at least one of Bluetooth protocols, low energy Bluetooth protocols, NFC protocols, RFID protocols, IR protocols, Wi-Fi protocols, ZigBee protocols, Z-Wave protocols, 6LoWPAN protocols, and/or other short range communication protocol. In various embodiments, the antenna 312, transmitter 304, and receiver 306 may be configured to communicate via one or more long range protocols, such as GPRS, UMTS, CDMA200, 1×RTT, WCDMA, GSM, EDGE, TD-SCDMA, LTE, E-UTRAN, EVDO, HSPA, HSDPA, Wi-Fi, Wi-Fi Direct, WiMAX, and/or the like. The user computing entity 20 may also include one or more network and/or communications interfaces 320 for communicating with various computing entities, such as by communicating data, content, information, and/or similar terms used herein interchangeably that can be transmitted, received, operated on, processed, displayed, stored, and/or the like.

In this regard, the user computing entity 20 may be capable of operating with one or more air interface standards, communication protocols, modulation types, and access types. More particularly, the user computing entity 20 may operate in accordance with any of a number of wireless communication standards and protocols. In a particular embodiment, the user computing entity 20 may operate in accordance with multiple wireless communication standards and protocols, such as GPRS, UMTS, CDMA200, 1×RTT, WCDMA, TD-SCDMA, LTE, E-UTRAN, EVDO, HSPA, HSDPA, Wi-Fi, WiMAX, UWB, IR protocols, Bluetooth protocols, USB protocols, and/or any other wireless protocol.

Via these communication standards and protocols, the user computing entity 20 can communicate with various other devices using concepts such as Unstructured Supplementary Service information/data (USSD), Short Message Service (SMS), Multimedia Messaging Service (MMS), Dual-Tone Multi-Frequency Signaling (DTMF), and/or Subscriber Identity Module Dialer (SIM dialer). The user computing entity 20 can also download changes, add-ons, and updates, for instance, to its firmware, software (e.g., including executable instructions, applications, program modules), and operating system.

According to one embodiment, the user computing entity 20 may include location determining aspects, devices, modules, functionalities, and/or similar words used herein interchangeably to acquire location information/data regularly, continuously, or in response to certain triggers. For example, the user computing entity 20 may include outdoor positioning aspects, such as a location module adapted to acquire, for example, latitude, longitude, altitude, geocode, course, direction, heading, speed, UTC, date, and/or various other information/data. In one embodiment, the location module can acquire information/data, sometimes known as ephemeris information/data, by identifying the number of satellites in view and the relative positions of those satellites. The satellites may be a variety of different satellites, including LEO satellite systems, DOD satellite systems, the European Union Galileo positioning systems, the Chinese Compass navigation systems, Indian Regional Navigational satellite systems, and/or the like. Alternatively, the location information/data may be determined by triangulating the apparatus's 30 position in connection with a variety of other systems, including cellular towers, Wi-Fi access points, and/or the like. Similarly, the user computing entity 20 may include indoor positioning aspects, such as a location module adapted to acquire, for example, latitude, longitude, altitude, geocode, course, direction, heading, speed, time, date, and/or various other information/data. Some of the indoor aspects may use various position or location technologies including RFID tags, indoor beacons or transmitters, Wi-Fi access points, cellular towers, nearby computing entities (e.g., smartphones, laptops) and/or the like. For instance, such technologies may include iBeacons, Gimbal proximity beacons, BLE transmitters, NFC transmitters, and/or the like. These indoor positioning aspects can be used in a variety of settings to determine the location of someone or something to within inches or centimeters.

The user computing entity 20 may also comprise a user interface device comprising one or more user input/output interfaces (e.g., a display 316 and/or speaker/speaker driver coupled to a processing element 308 and a touch interface, keyboard, mouse, and/or microphone coupled to a processing element 308). For example, the user interface may be configured to provide a mobile application, browser, interactive user interface, dashboard, webpage, and/or similar words used herein interchangeably executing on and/or accessible via the user computing entity 20 to cause display or audible presentation of information/data and for user interaction therewith via one or more user input interfaces. Moreover, the user interface can comprise or be in communication with any of a number of devices allowing the user computing entity 20 to receive information/data, such as a keypad 318 (hard or soft), a touch display, voice/speech or motion interfaces, scanners, readers, or other input device. In embodiments including a keypad 318, the keypad 318 can include (or cause display of) the conventional numeric (0-9) and related keys (#, *), and other keys used for operating the user computing entity 20 and may include a full set of alphabetic keys or set of keys that may be activated to provide a full set of alphanumeric keys. In addition to providing input, the user input interface can be used, for example, to activate or deactivate certain functions, such as screen savers and/or sleep modes. Through such inputs the user computing entity 20 can capture, collect, store information/data, user interaction/input, and/or the like.

The user computing entity 20 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, RRAM, SONOS, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. The volatile and non-volatile storage or memory can store databases, database instances, database management system entities, information/data, applications, programs, program modules, scripts, source code, object code, byte code, compiled code, interpreted code, machine code, executable instructions, and/or the like to implement the functions of the user computing entity 20.

### Exemplary Networks

In one embodiment, any two or more of the illustrative components of the system architecture 100 of FIG. 1 may be configured to communicate with one another via one or more networks 30. The networks 30 may include, but are not limited to, any one or a combination of different types of suitable communications networks such as, for example, cable networks, public networks (e.g., the Internet), private networks (e.g., frame-relay networks), wireless networks, cellular networks, telephone networks (e.g., a public switched telephone network), or any other suitable private and/or public networks. Further, the networks 30 may have any suitable communication range associated therewith and may include, for example, global networks (e.g., the Internet), MANs, WANs, LANs, or PANs. In addition, the networks 30 may include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, satellite communication mediums, or any combination thereof, as well as a variety of network devices and computing platforms provided by network providers or other entities.

### Example System Operation

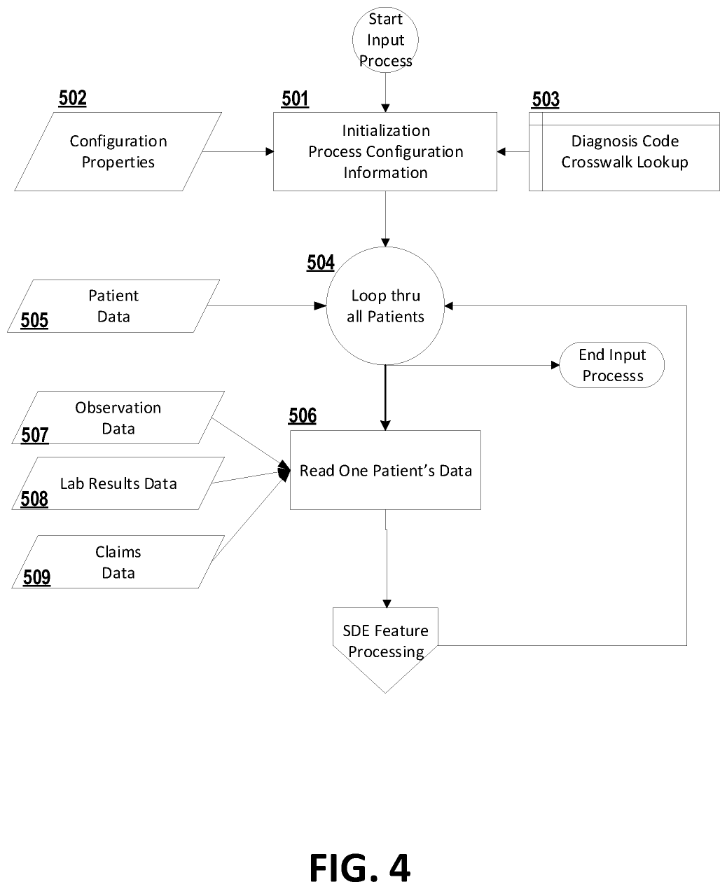

The pre-processing methodology of an example system operation is discussed below in reference to FIGS. 4-8G. The example operation of the overall system is discussed in terms of data inputs, data pre-processing, and pre-processing output data to serve as an input for further analytics (e.g., as a part of severity modeling). The configurations discussed herein are specifically provided for pre-processing cancer stage data to generate consistent output data sets which can subsequently be utilized in a plurality of analytic applications including, but not limited to, computer-based modeling such as severity models for generating granular data indicative of the cost of treating a patient's cancer.

### Data Input

Certain embodiments encompass pre-processing configurations to provide pre-processing of data, such as claims data (e.g., data submitted to request reimbursement for medical services/products from a payer), and/or non-claims data, such as observation data (e.g., physician notes, prescription data, lab data, EHR data, data submitted during a prior authorization process, and/or the like) and/or other types of clinical data to enable ingestion of the pre-processed data into subsequent analytics including, but not limited to computer-based modeling such as machine-learning based models for determining severity attributes of a patient's cancer. In certain embodiments, the data input comprises a plurality of data records (e.g., claims data records, observation data records, other clinical data records, and/or the like), with each data record comprising structured data embodied as a plurality of data fields each having relevant substantive data stored therein.

In certain embodiments, the input data corresponds with a particular patient (which may be identified by any of a variety of unique identifiers, such as a patient name, a patient unique user identifier, a unique identifier associated with the patient (e.g., a social security number), and/or the like. Moreover, the input data may be associated with one or more medical conditions, medical treatments, and/or the like, and such data may comprise a unique identifier indicative of the medical condition and/or medical treatment to which a particular data record (or other data grouping) applies. The unique identifier corresponding with the medical condition and/or medical treatment may be a universally recognized identifier taken from a universally known code-base, such as ICD codes (e.g., ICD-9 codes, ICD-10 codes, and/or the like). In other embodiments, a unique identifier corresponding with a medical condition may be a proprietary code identified within a proprietary code ontology. In certain embodiments, the pre-processing systems as discussed herein comprise one or more lookup tables for converting certain codes into codes usable by the pre-processing system (e.g., converting proprietary codes into ICD-10 codes, and/or the like). It should be understood that the one or more lookup tables may comprise lookup tables provided as a part of an initial setup of the pre-processing system and/or one or more lookup tables provided by an end-user of the system (e.g., so as to enable the pre-processing system to operate properly with proprietary coding systems). As discussed herein, certain pre-processing methodologies are specifically configured for generating model input data sets based on a particular medical condition, and accordingly various embodiments as discussed herein are configured to apply a particular pre-processing methodology to a plurality of data records within a data input that all have a common, shared indicator of a particular medical condition (e.g., a particular cancer type). For example, all data records having a breast cancer indicator may be analyzed in accordance with a relevant pre-processing methodology, all data records having a colon cancer indicator may be analyzed in accordance with a relevant pre-processing methodology, and/or the like.

As indicated above, input data may be provided as one or more data records. In certain embodiments, each data record may correspond to a medical interaction with a patient. For example, such medical interactions may comprise an in-person visit between the patient and a medical professional, a virtual visit between the patient and a medical professional, a pharmaceutical prescription pick-up, a specific interaction with the patient during an in-patient stay at a medical facility, a medical device provided to the patient (e.g., as a prescription, as a part of an in-patient visit, and/or the like), and/or the like.

In certain embodiments, the pre-processing system is configured to intake a plurality of data record types, such as claims data records, member data records, observation data records, other clinical data records (e.g., lab data records), and/or the like. The input data records may be provided with metadata defining one or more characteristics of the data record, thereby facilitating pre-processing thereof before providing the pre-processed data to one or more downstream analytics (such as a machine-learning severity model). Moreover, the pre-processed data may be stored and later merged with additional data to collectively define training data for one or more of the downstream analytics. For example, the pre-processed data may later be merged with externally-provided results data that provides an indication of one or more objective severity attributes (e.g., total incurred costs for treatment; itemized incurred costs for treatment; and/or the like) and the merged data may be utilized as training data for a machine-learning based severity model so as to enable generation of machine-learning severity models capable of generating accurate and precise outputs. The metadata may be provided together with the data file, or the metadata may be provided in a separate data file linked with (e.g., via matching reference identifiers) the underlying data record. The metadata may comprise data identifying, for example, a data record location (e.g., the storage location of the underlying data record), date formats within the data record, one or more dictionaries and/or reference tables for enabling automated processing of data elements within the data record.

The pre-processing systems are configured for filtering data records from a received collection of input data records such that removed data records are not included within the generated model input data that is generated as output of the pre-processing systems and passed to downstream analytics as input thereto. Therefore, the removed data records are not provided to the downstream analytics and therefore do not influence training of the downstream analytics (in the case of machine-learning based models) and/or do not influence the resulting outputs generated by processing the generated model input data via the downstream analytics. Accordingly, the pre-processing system is configured to execute one or more data record validation processes, which are configured to ensure that the input data records are provided in a processable data format, comprise necessary data types, and/or the like. For example, the data record validation processes may be configured to ensure that each data record comprises one or more date fields (e.g., so as to ensure that only data records having corresponding dates falling within a defined date range are included so as to enable chronological analysis of data records), and/or the like. In certain embodiments, the data record validation process is user configurable, and may comprise one or more configurable settings, such as defining pre-processing output storage locations, pre-processing log file storage locations, and/or the like.

The pre-processing data record validation processes are further configured to ensure that proper data and proper data records are provided as input before executing one or more pre-processing filtering processes. For example, the pre-processing data record validation processes of certain embodiments are configured to ensure that patient-identifying data records are provided along with one or more observation data records and/or other clinical data records (e.g., lab data records) and/or claims data records. Accordingly, the pre-processing system is configured to intake patient-identifying data records along with one or more observation data records, and/or claims data records. The pre-processing system performs such intake at a patient-level, such that observation records and/or claims records provided as input together with the patient identifying data record are associated with the patient identifying data record.

To intake the data, the pre-processing system reads in patient identifying data records to determine which observation data records and/or claims data records are to be read for pre-processing. As mentioned, the observation data records and/or claims data records comprise a unique identifier corresponding to the patient, thereby enabling the patient data record to be matched with the observation data records and/or claims data records when reading such data records.

Observation data records relating the patient data record are read from an observation input file comprising a plurality of observation data records. As noted above, relevant observation data records are identified based at least in part on a patient identifier stored within the observation data records. Examples of observation data elements that may be contained within an observation data record encompass measurements of a patient's Body Mass Index (BMI), systolic and diastolic blood pressure (SBP and DBP), and/or the like. In accordance with certain embodiments, each observation data record contains only a single observation measurement, and accordingly a single patient may have a plurality of observation data records generated within a single day. However, it should be understood that in certain embodiments, an observation data record may contain a plurality of observation measurements (e.g., a series of physician notes, and/or the like) for a patient, and such observation data records may be subdivided as necessary to enable further pre-processing as discussed herein.

Lab data records relating to the patient data record are read from a lab results input file comprising a plurality of lab data records. As noted above, relevant lab data records are identified based at least in part on a patient identifier stored within the observation data records. Examples of lab data elements that may be contained within a lab data record encompass creatinine measurements, hemoglobin results, biomarker mutation results (e.g., for detecting RAS/KRAS/NRAS mutations), other oncology-related laboratory testing, and/or the like. In accordance with certain embodiments, each lab data record contains only a single lab measurement, and accordingly a single patient may have a plurality of lab data records generated within a single day. However, it should be understood that in certain embodiments, a lab data record may contain a plurality of lab measurements (e.g., results from a plurality of lab tests performed on a patient during a single day) for a patient, and such lab data records may be subdivided as necessary to enable further pre-processing as discussed herein.

Claims data records relating to the patient data record are read from one or more claims data sources. In certain embodiments, the claims data records may comprise claim data elements indicative of a medical procedure and/or a medical condition of the corresponding patient. Such data may be provided in the form of codes (e.g., ICD-10 codes). In certain embodiments, the claims data records may be utilized as discussed herein to generate derived observation data records indicative of certain clinical observations and/or to resolve conflicts within and/or between observation data records for a particular patient (e.g., during cancer stage analysis as discussed herein).

FIG. 4 is a flowchart illustrating an example process for intaking and processing data. As shown therein, the pre-processing system initializes the data input process as shown at Block 501, by ingesting configuration properties (shown at Block 502) for execution of the data input process. Moreover, as needed, one or more lookup tables may be referenced during initialization (e.g., if diagnosis codes are to be translated between coding structures, such as between a proprietary diagnosis code set and ICD-10 codes) as indicated at Block 503.

The process is performed one patient at a time, for all patients for which patient data is provided (as indicated at Blocks 504-505 and the looping structure of the flowchart). For each patient, observation data records, other clinical data records, claims data records, and/or any other data records relevant to a member are read, as indicated at Blocks 506-509.

In certain embodiments, each of the patient data records, the clinical data records, the lab data records, and/or the claims data records may be stored within a relational database, with each data record stored separately, and having one or more data elements stored therein that may be utilized to identify relationships between data records (e.g., via a unique patient identifier). However, it should be understood that other database structures may be utilized in certain embodiments.

In example embodiments, the patient data files comprise a unique patient identifier and a date of birth for the patient. The patient data files may comprise additional data relevant to a patient, such as contact information (e.g., a phone number, an email address, a home address, a mailing address, and/or the like), insurance information (e.g., identifying a health insurance provider, health insurance membership information, and/or the like).

Observation data records of certain embodiments comprise a unique patient identifier to enable association with a patient data record, a unique record identifier that may be utilized to quickly identify a particular observation record, a concept type identifier indicating the type of data stored within the observation data record (e.g., a clinical reading, a diagnostic test, and/or the like), a start date for the observation, an observation value (identifying the substantive measurement of the observation, such as indicating a positive test result for a particular biomarker mutation, an indication of a particular cancer stage, and/or the like), a concept condition identifier, such as a condition for which the observation value is provided (e.g., in oncology-related measurements, the concept condition identifier indicates a cancer type), a data source identifier indicative of the source from which the observation data record is retrieved (e.g., a patient's electronic medical record, a pre-authorization request data record submitted to an insurance payer, and/or the like), and/or the like.

Lab data records of certain embodiments comprise a unique patient identifier to enable association with a patient data record, a unique record identifier that may be utilized to quickly identify a particular lab data record, a code type (that may be utilized to indicate the taxonomy of the additional data elements provided in the lab data record, such as indicating whether the lab results data record utilizes LOINC coding structures, a proprietary coding structure of a particular lab, and/or the like), a code (e.g., a LOINC code indicative of a lab test performed), a start date for the lab result, a data element indicative of the lab result (e.g., a numeric data element, a non-numeric data element, a binary data element, and/or the like), a results unit identifier (indicative of the units relevant to the lab result itself), a data source identifier indicative of the source from which the observation data record is retrieved (e.g., a patient's electronic medical record, a pre-authorization request data record submitted to an insurance payer, and/or the like), and/or the like.

Claims data records of certain embodiments comprise a unique patient identifier enabling association with a patient data record, a code type (that may be utilized to indicate the taxonomy of the additional data elements provided in the claims data record, such as indicating whether the claims data record utilizes ICD-10 codes, ICD-9 codes, or another diagnosis code taxonomy), one or more diagnosis codes, a first date of service associated with the claim, a last date of service associated with the claim, and/or a unique record identifier that may be utilized to quickly identify a particular claim data record.

### Data Pre-Processing

As discussed herein, the data pre-processing methodologies for cancer stage, when executed by a pre-processing system that may embody a portion of a management computing entity, may be configured for pre-processing observation data records and/or claims data records before passing the pre-processed data to a downstream analytic to be utilized as data input to the downstream analytic. Cancer stage data sourced from clinical observations provides information regarding the extent of a patient's cancer; this clinical observation-based data cannot be obtained from claims data. This information can be used to better understand the severity of the cancer, as well as the associated estimated treatment costs (e.g., via a downstream machine-learning based severity model). In certain embodiments, cancer stage observation data records may be collected and/or generated during a prior authorization process and/or by collecting data from a patient's EMR.

The pre-processing methodology, when executed by a pre-processing system, discussed herein is configured to output one or more data records having consistent cancer stage indications for a single patient, over a defined time frame. The output of one or more data records may be provided as a part of a model input data set to be passed to a downstream analytic such as a machine-learning based severity model. The model input data set generated by the pre-processing methodology may be embodied as a flat file or a series of separate files. By providing the model input data set as a flat file, the data within each of the contained data records is accessible without additional latency associated with providing separate data files each corresponding to a single data record.

The pre-processing system thus identifies clinically invalid and/or conflicting cancer stage observations data records (e.g., between condition-specific observation data records such as cancer stage observation data records having a shared/common cancer type identifier), such that only consistent data records constituting clinically valid and non-conflicting findings are provided to subsequent analytics, including, but not limited to computer-based modeling such as machine-learning based severity modeling. These clinically invalid and/or conflicting cancer stage observations data records may be identified via one or more data content filter criteria, which may be configured to rely on substantive contents of one or more data records to identify those clinically invalid and/or conflicting cancer stage observations. Moreover, as discussed herein, preliminary filter criteria, such as data source filter criteria may be implemented in certain embodiments to eliminate certain data records (e.g., by eliminating data records received from a data source not indicated as eligible for providing data input). Other preliminary filter criteria, such as date-based filter criteria eliminating data records not falling within a defined date range in question, may additionally be implemented as discussed herein to further decrease the total number of data records under consideration.

As discussed herein, claims data, including diagnostic data such as diagnosis codes provided as a part of claims data, may be utilized to address certain identified inconsistencies between observation data records, such as by determining whether any secondary malignancies are present that may indicate that an advanced stage of cancer is present (thereby eliminating a potential inconsistency between observation data records indicating various cancer stages by eliminating the lower-stage observation record). Consistent cancer stage indications as discussed herein entail having an unchanging cancer stage indication over an entire time period (e.g., Stage III cancer over the entire time frame in question) or having changing cancer stage indications that satisfy oncology-specific clinical standards. For example, those rules may specify that a cancer stage may advance to a more severe cancer stage over a defined time frame, but the cancer stage may not retreat to a less severe cancer stage over the defined time frame. Specific rules for defining consistent cancer stage data may be provided for specific cancer types, and example rules are discussed in greater detail herein with reference to specific cancer types. Specifically, rules for defining consistent cancer stage data are discussed herein with reference to Small Cell Lung Cancer (SCLC); Breast Cancer, Colon Cancer, Rectal Cancer, and Non-Small Cell Lung Cancer (NSCLC) (collectively referred to as BCRN); and Prostate Cancer. The described rules identify and resolve intra-date conflicts encompassing a single date with multiple, conflicting observation records each indicative of a different relevant cancer stage, as well as inter-date conflicts encompassing multiple, conflicting observation records each indicative of a different relevant cancer stage reported across an entire time frame. The pre-processing system as discussed herein identifies conflicts and resolves these conflicts by either removing all conflicting information or querying claims data to provide supportive evidence that a metastatic stage observation should be retained and included in data provided to downstream analytics (despite the presence of otherwise conflicting observation data).

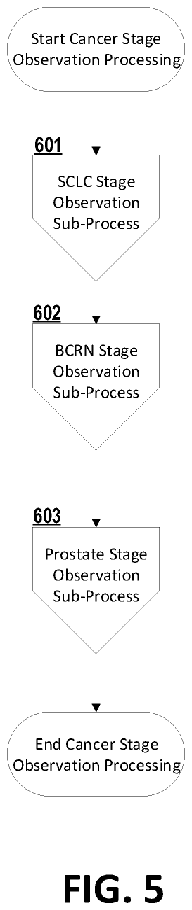

As illustrated in FIG. 5, the pre-processing system may determine the relevance of particular consistency rules to certain observation data records based on data included in the observation data records indicating a cancer type. These rules may be applied in series, such that the pre-processing model determines the relevance of particular rules in series, for example, by first determining whether rules relevant to SCLC observations are relevant to particular data records (as shown at Block 601), then determining whether rules relevant to breast, colon, rectal, or NSCLC observations are relevant to particular data records (as shown at Block 602), then determining whether rules relevant to prostate cancer are relevant to particular data records (as shown at Block 603). It should be understood that other rules relevant to other cancer types may be considered in series as well. In other embodiments, rules relevant to specific cancer types may be provided and considered in separate models, and the pre-processing system may first identify a relevant model for specific data records before executing rule-based analysis of the observation data records in accordance with the selected pre-processing model. Moreover, although the following discussion presents these models as being executed in a specific example order, it should be understood that the order of executing the various models may be provided in any model execution order.

### Small Cell Lung Cancer

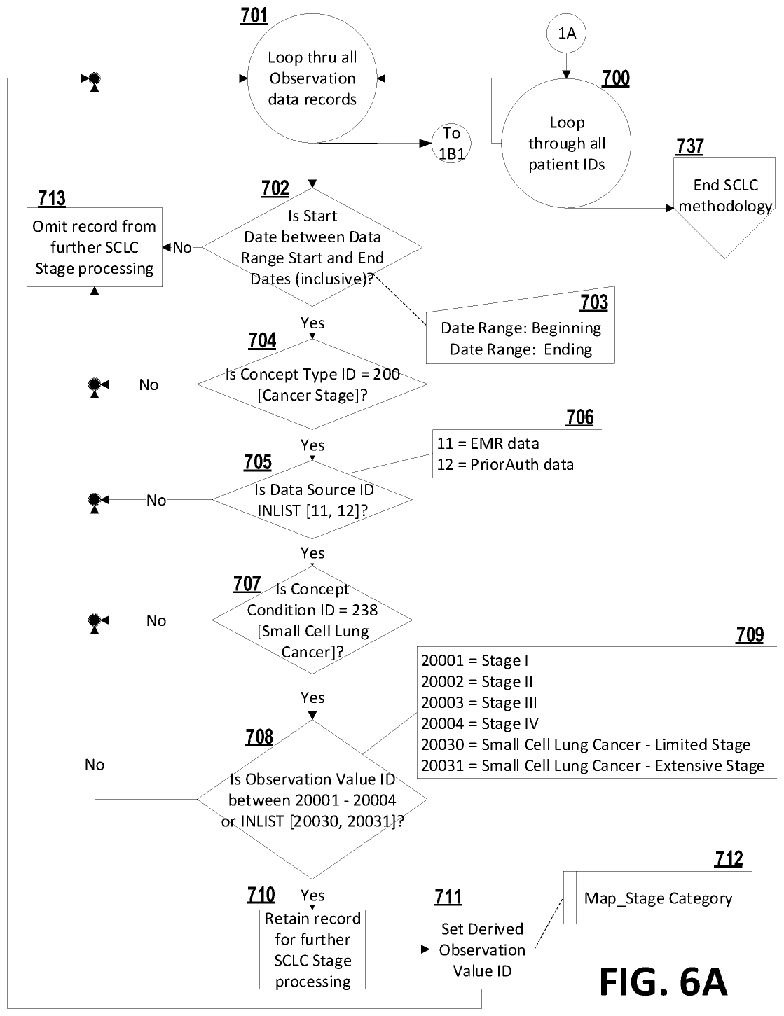

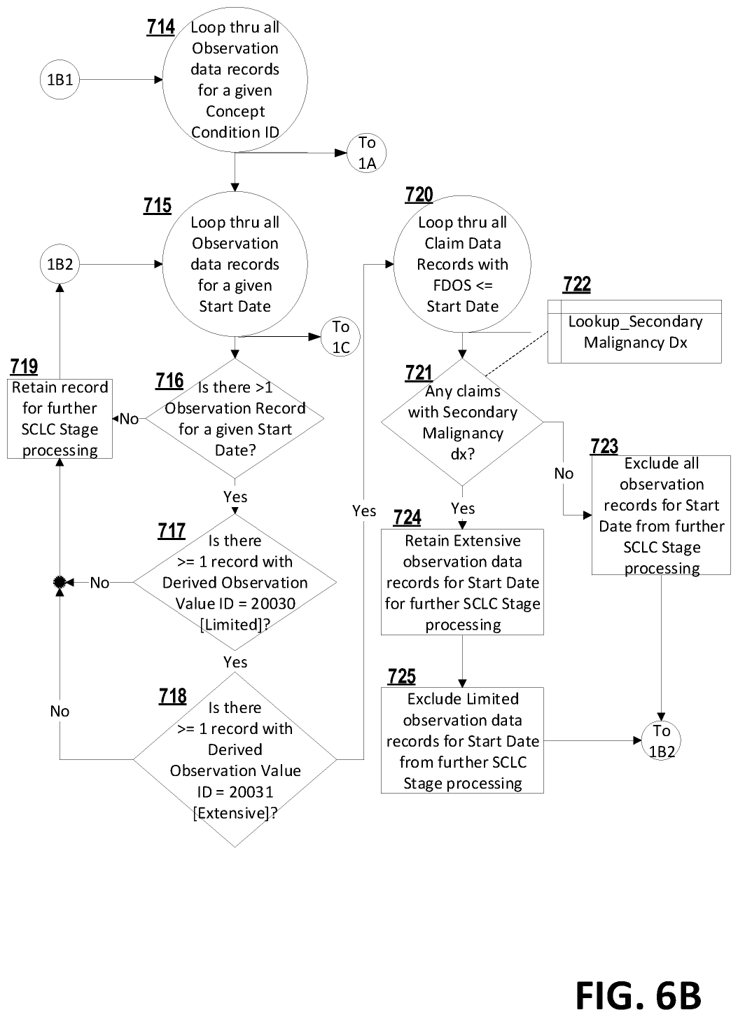

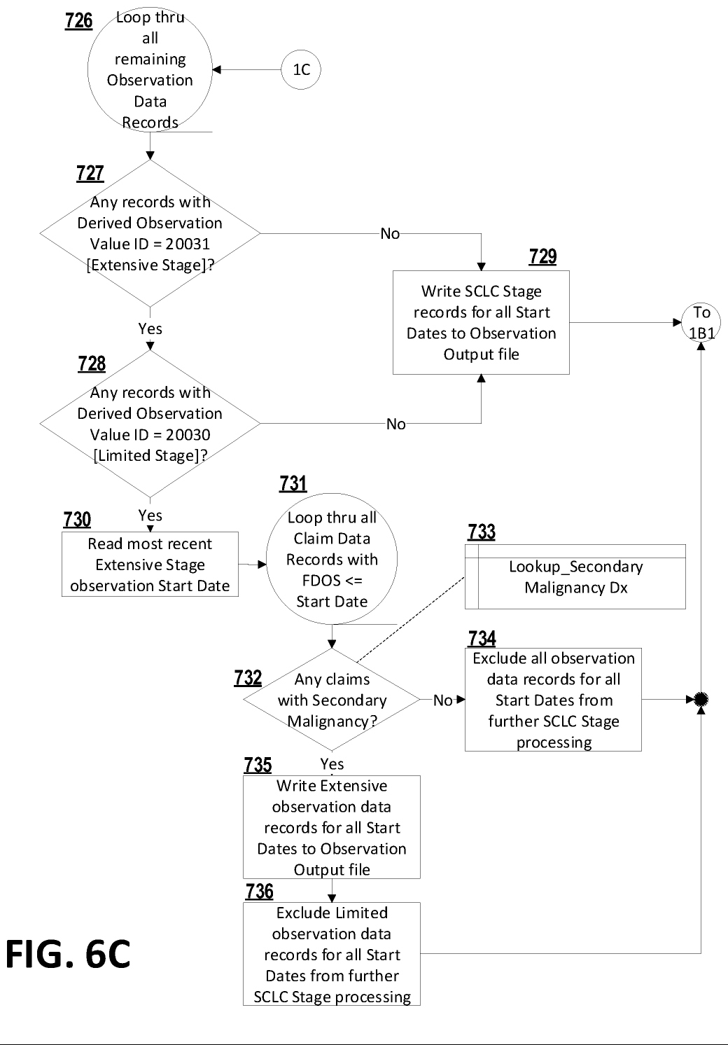

FIGS. 6A-6C collectively illustrate an example flowchart of a SCLC pre-processing methodology that may be executed by a pre-processing system according to one embodiment. As shown at Block 700, the methodology reflected in the flowchart may be performed for all patients separately (the pre-processing system executing the described methodology proceeding to analyze data records for a single patient before moving to the next patient). Moreover, the described steps reflected within FIGS. 6A-6C may be performed by looping through all records associated with the patient's unique identifier (as reflected at Block 701), so as to identify potentially conflicting and/or otherwise inconsistent data records or SCLC stage observations associated with the patient. As illustrated in FIGS. 6A-6C, the pre-processing system executing the pre-processing methodology applies a plurality of filtering steps in sequence, such that a data record analyzed in accordance with any one of the described filtering steps has already been determined to satisfy all of the previously applied filtering steps. This sequential application of filtering steps enables complex filtering steps requiring simultaneous satisfaction of a plurality of factors to be applied in a simple manner.

As shown at Block 702, the pre-processing system executing the SCLC pre-processing methodology identifies observation data records having start dates within a range of dates identified as defining a relevant time frame for analysis. The date range may be defined within the configuration data for the pre-processing system in general and may be defined in terms of a beginning date and an ending date for the date range (as shown at Block 703). Those records having dates that do not fall within the relevant date range are excluded from further analysis, as indicated at Block 713.

While executing the SCLC pre-processing methodology, the pre-processing system continues by identifying data records having a concept type identifier indicating the observation data record relates to cancer stage, as indicated at Block 704. Those records that do not contain data elements indicating the observation data record relates to cancer stage data are excluded from further analysis, as indicated at Block 713.

The SCLC pre-processing methodology (executed by the pre-processing system) continues by identifying data records indicating the data record source is one of one or more permitted data sources, as indicated at Block 705. For example, the pre-processing system executing the SCLC pre-processing methodology may be configured to maintain observation data records obtained from a patient's EMR and/or from preauthorization request data (as indicated at Block 706). In certain embodiments, the SCLC pre-processing methodology is configured to cause the pre-processing system to accept data records from a single permitted data source. Those observation data records indicated as received from an unpermitted data source are excluded from further analysis, as indicated at Block 713.

The SCLC pre-processing methodology (executed by the pre-processing system) continues by identifying data records indicating the data record relates to SCLC, as indicated at Block 707. Subsequent pre-processing methodologies (as indicated in FIGS. 6B and 6C) implement SCLC-specific stage rules for identifying potentially conflicting or inconsistent data records and for identifying resolutions for identified data conflicts. Observation data records that do not relate to SCLC are excluded from further SCLC stage processing, as indicated at Block 713 (however such data records may be analyzed in accordance with other pre-processing methodologies as discussed herein if those data records are identified as relevant to those other pre-processing methodologies).

The SCLC pre-processing methodology, when executed by the pre-processing system, is specifically configured for identifying conflicts between data records based on differences in identified cancer stages. Accordingly, as indicated at Block 708, the SCLC pre-processing methodology determines whether an observation value identifier associated with each data record matches one of a defined number of available observation values relating to relevant cancer stage identifiers for SCLC. As shown at Block 709, which provides a list of relevant observation value identifiers, the SCLC stage pre-processing methodology causes the pre-processing system to determine whether a data record comprises an observation value identifier indicative of Stage I cancer, Stage II cancer, Stage III cancer, Stage IV cancer, Limited Stage SCLC, or Extensive Stage SCLC. Those data records that do not contain observation value identifiers indicative of any of the acceptable observation value identifiers are excluded from further SCLC stage processing, as indicated at Block 713.

After identifying records satisfying each of the filtering rules discussed in reference to Blocks 702-709, those records are retained for further analysis, as indicated at Block 710, and the remaining data records are modified by the addition of a derived observation value identifier field (and corresponding derived observation value identifier) as indicated at Block 711, which is populated based on a lookup table as indicated at Block 712. The derived observation value identifier may be determined based at least in part on the observation value identifier, and the lookup table maps observation value identifiers to corresponding derived observation value identifiers. The derived observation value identifiers of an example embodiment are simplified relative to the number of available observation value identifiers. Specifically, the derived observation value identifier may be selected from two available derived observation value identifiers (limited stage SCLC or extensive stage SCLC). Stage I, Stage II, Stage III, and limited stage SCLC observation value identifiers will all map to the limited stage SCLC derived observation value identifier; while Stage IV and extensive stage SCLC observation value identifiers will map to the extensive stage SCLC derived observation value identifier.

With reference specifically to FIG. 6B, for those records identified as having a concept condition identifier indicative of SCLC and a valid SCLC stage (which were retained at step 710), the pre-processing system executing the SCLC pre-processing methodology continues applying various filtering analyses (as indicated at Block 714) for identifying and rectifying any identified conflicts between data records. According to the SCLC pre-processing methodology, the pre-processing system first identifies intra-date conflicts, as discussed in reference to Blocks 715-723. Accordingly, as indicated at Block 715, the SCLC pre-processing methodology (when executed by the pre-processing system) identifies all observation records having a single start date to identify intra-date conflicts between data records associated with a same date (e.g., having a data element identifying a shared start date). If, according to the SCLC pre-processing methodology, the pre-processing system identifies only one observation record for a particular date (as shown at Block 716), the observation record is retained for further analysis, as indicated at Block 719. However, if more than one observation record is identified for a particular date, those identified records are further analyzed to determine whether they should be retained for further analysis. As shown at Block 717, the pre-processing system determines whether one or more observation records for a particular date have a derived observation value identifier indicative of limited stage SCLC. If no observation records have a derived observation value identifier of a limited stage SCLC (indicating that all records have a derived observation value of extensive stage SCLC), the records for that date are retained, as indicated at Block 719.

However, if one or more observation records have a derived observation value identifier indicative of a limited stage SCLC indication, the SCLC pre-processing methodology, when executed by the pre-processing system, next determines whether there are also one or more observation records having a derived observation value identifier of an extensive stage SCLC on the same date, as indicated at Block 718. If no observation records are identified as having a derived observation value identifier of extensive stage SCLC (indicating that all records have a derived observation value of limited stage SCLC), the records for that date are retained for further analysis, as indicated at Block 719 (e.g., for severity modelling, as discussed in greater detail herein). However, if one or more observation records are identified as having a derived observation value identifier of an extensive stage SCLC and a derived observation value identifier of limited stage SCLC on the same date, the intra-date conflict analysis of the SCLC pre-processing methodology continues, as indicated at Block 720, to retrieve relevant claims data, such that diagnosis codes may be utilized to address certain identified intra-date conflicts. The relevant claims data records are those claims data records having a first date of service on or before the first date having both extended stage and limited stage SCLC derived observation value identifiers. Utilizing a lookup table mapping specific diagnosis codes to an indication of whether those diagnosis codes are indicative of a secondary malignancy (the lookup table reflected at Block 722), the SCLC pre-processing methodology, when executed by the pre-processing system, determines whether any claims data records are indicative of a secondary malignancy, as indicated at Block 721. If no secondary malignancies are identified, then all observation records for that particular date are excluded from further SCLC stage processing, as indicated at Block 723. However if a secondary malignancy is identified, the observation records having an extensive stage SCLC derived observation value identifier for that date are retained (as indicated at Block 724) and any observation records for that date having a limited stage SCLC derived observation value identifier are excluded from further SCLC stage processing, as indicated at Block 725.

Although not specifically reflected within the flowchart of FIG. 6B, the SCLC pre-processing methodology may be further configured to cause the pre-processing system to implement one or more tiebreaker rules for eliminating entirely duplicative observation data records occurring on a single date. For example, tiebreaker rules may prioritize data generated by a particular data source, such that when duplicative data are available for a particular date, and one duplicative observation data record is generated by a preferred data source, the data record generated by the preferred data source is retained, and other observation data records for that date are eliminated. As yet another example, a tiebreaker rule may prioritize data records having a lower unique record identifier (thereby prioritizing the “first” generated data record). Data records having higher unique record identifiers (generated after the first-generated data record) may be eliminated. It should be understood that multiple tiebreaker rules may be implemented in certain embodiments, utilizing a hierarchy of tiebreaker rules that are applied in hierarchical sequence until duplicative data records are eliminated.

FIG. 6C is a flowchart specifically focusing on subprocesses for identifying and rectifying inter-date conflicts identified between observation data records. As noted at Block 726, the processes for identifying and rectifying inter-date conflicts identified between observation data records is performed for those observation data records that have not otherwise been excluded, for example, through the processes discussed in reference to FIGS. 6A-6B. As indicated at Blocks 727-728, the SCLC pre-processing methodology causes the pre-processing system to determine whether there is a mixture of extensive and limited stage data records associated with a particular patient for a particular time frame. As reflected at Blocks 727-728, such a determination may be performed sequentially, by first determining whether any observation data records have extensive stage SCLC derived observation value identifiers, as reflected at Block 727; if none are present (indicating only limited stage SCLC derived observation value identifiers were identified), the process proceeds as reflected at Block 729 and the SCLC stage records for all start dates are retained. If observation data records for extensive stage SCLC derived observation value identifiers are present, next it is determined whether any observation data records have limited stage SCLC derived observation value identifiers, as reflected at Block 728. If no limited stage SCLC derived observation value identifiers were identified (indicating only extensive stage SCLC derived observation value identifiers were identified), the process proceeds as indicated at Block 729 and the SCLC stage records for all start dates are retained. In summary, the sub-processes reflected at Blocks 727-729 determine whether only observation data values with limited stage SCLC derived observation value identifiers are present for a patient within a particular time frame, or whether only observation data values with extensive stage SCLC derived observation value identifiers are present for a patient within the particular time frame. If only extensive stage or only limited stage derived observation value identifiers are present for the patient during the particular time frame, all SCLC stage data records are retained. However, if a mixture of extensive stage and limited stage derived observation value identifiers are present, the inter-date conflict analysis continues, as reflected at Block 730, by determining the start date of the most recent observation data record having an extensive stage SCLC derived observation value identifier. Claims data records relevant to the patient and having a first date of service on or before the identified start date of the most-recent observation data record having an extensive stage SCLC derived observation value identifier are retrieved, as reflected at Block 731, and each retrieved claims data record is reviewed to determine whether any claims reflect a secondary malignancy diagnosis code as reflected at Block 732 (with reference to a lookup table correlating diagnosis codes with an indication of secondary malignancy, as reflected at Block 733).

If no secondary malignancy is identified with reference to the retrieved claims data, all observation data records for all start dates are omitted from SCLC stage processing, as reflected at Block 734. However, if at least one diagnosis code within a reviewed claims data record is reflective of a secondary malignancy, the observation data records having extensive stage SCLC derived observation value identifiers for all start dates are retained, as reflected at Block 735, and all observation data records having limited stage SCLC derived observation value identifiers for all start dates are omitted from SCLC stage processing, as reflected at Block 736 (and are thus excluded from the severity modeling as discussed in greater detail herein).

The remaining retained SCLC stage records after executing the SCLC pre-processing methodology are consistent for a particular patient, such that the retained data records do not indicate a changing SCLC stage for the particular patient either within a particular date, or across multiple dates within a relevant time period. Moreover, any conflicts identified (that could otherwise lead to inconsistent data for the patient) are rectified, for example, by eliminating data records for which a conflict cannot otherwise be eliminated, or by referencing additional data (e.g., claims data) to rectify certain conflicts, if appropriate. The remaining SCLC stage observation data records are then available for use by downstream analytics, including, but not limited to, computer-based modeling such as machine-learning based severity models. Moreover, it should be understood that all of the observation data records (including those excluded from further analysis) may be retained within a log data file for later auditing.

As mentioned, the SCLC pre-processing methodology is repeated for all patients for whom patient identifiers are reflected within input data, before the SCLC pre-processing methodology ends, as reflected at Block 737 on FIG. 6A.

### Breast Cancer, Colon Cancer, Rectal Cancer, and Non-Small Cell Lung Cancer

Next, the pre-processing system continues by implementing a Breast, Colon, Rectal, and Non-Small Cell Lung Cancer (BCRN) pre-processing methodology. These multiple cancer-specific pre-processing methodologies may be executed in series or in parallel. As mentioned herein, the order in which the multiple pre-processing methodologies are executed may be changed, as the results of one pre-processing methodology are not dependent on any of the other pre-processing methodologies discussed herein (e.g., the BCRN pre-processing methodology is not dependent on the SCLC pre-processing methodology).

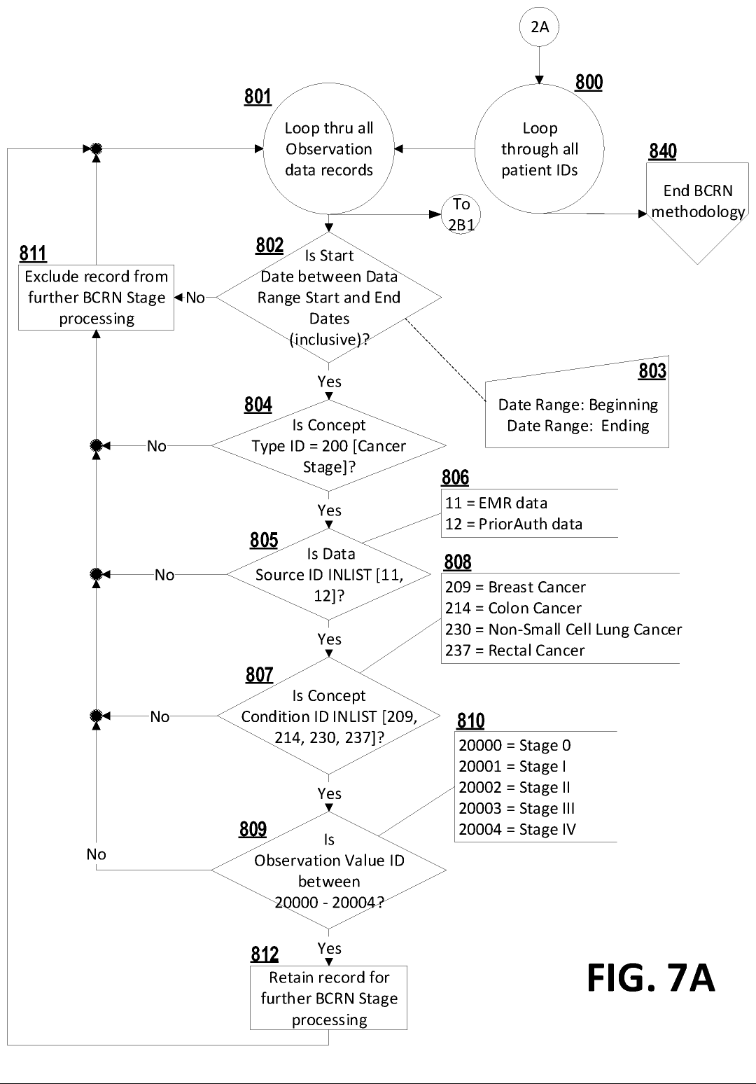

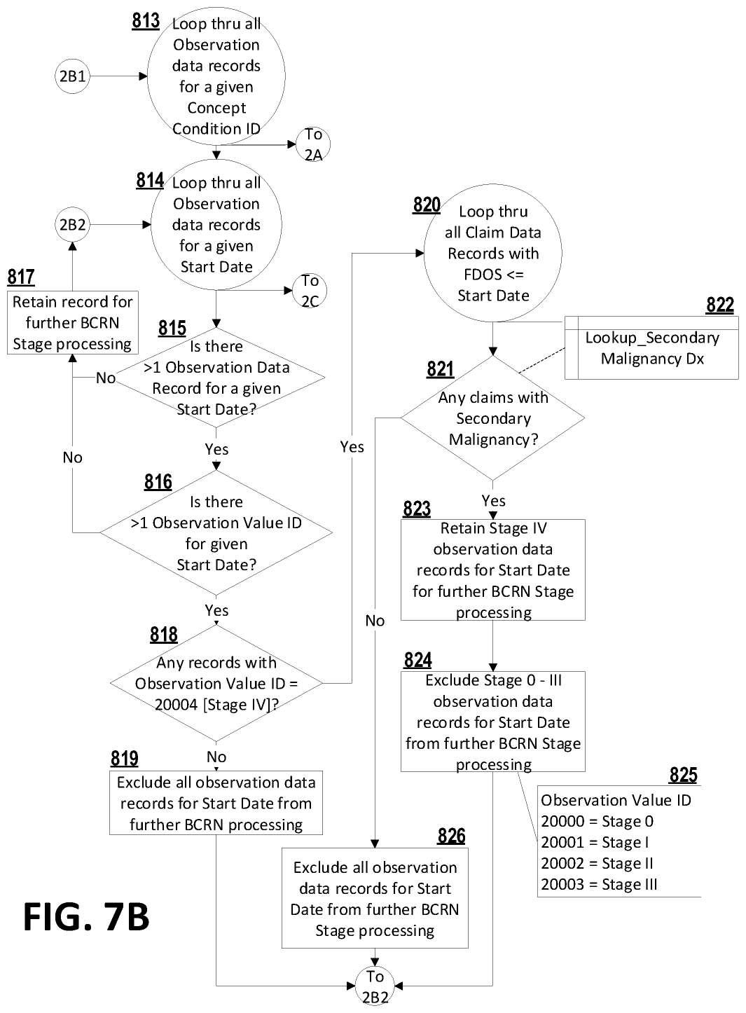

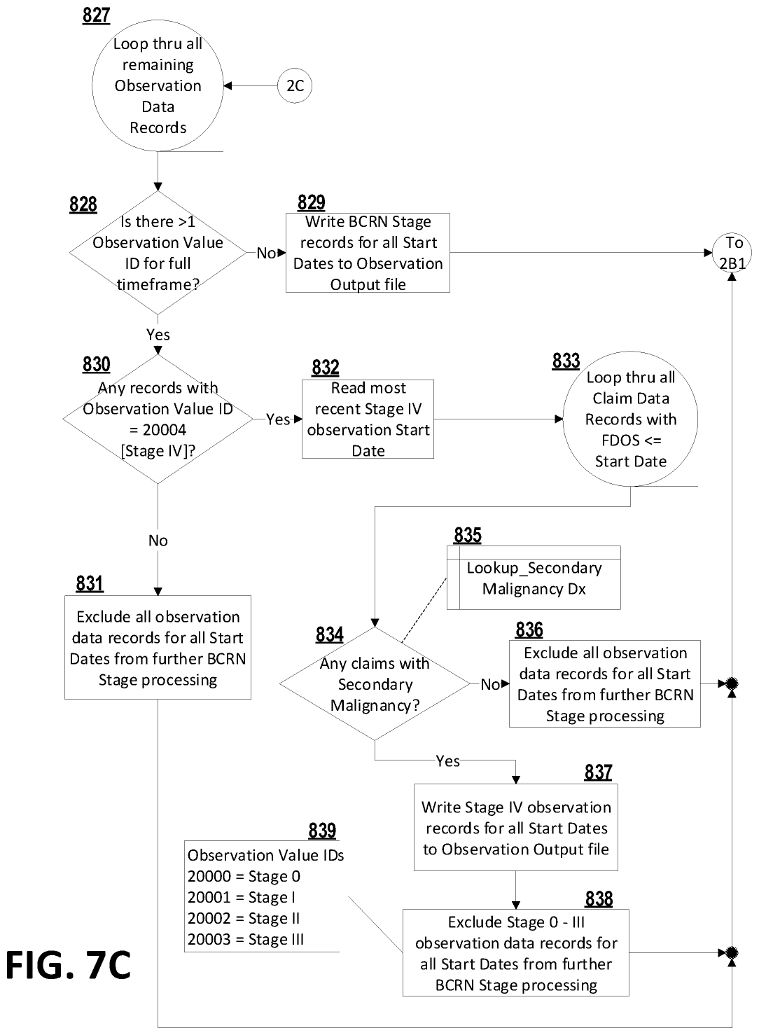

FIGS. 7A-7C collectively illustrate an example flowchart of a BCRN pre-processing methodology according to one embodiment. Clinically, each of breast cancer, colon cancer, rectal cancer, and Non-Small Cell Lung Cancer (NSCLC) are subject to analogous cancer stage categorizations, and accordingly observation data records corresponding to each of these cancer types may be pre-processed utilizing an analogous pre-processing methodology as discussed in reference to FIGS. 7A-7C. As shown at Block 800, the methodology reflected in the flowchart may be performed by the pre-processing system for all patients separately (the described methodology proceeding to analyze data records for a single patient before moving to the next patient). Moreover, the described steps reflected within FIGS. 7A-7C may be performed by looping through all records associated with the patient's unique identifier (as reflected at Block 801), so as to identify potentially conflicting and/or otherwise inconsistent data records or BCRN observations associated with the patient. Moreover, although the pre-processing methodology described in reference to FIGS. 7A-7C is discussed generally with respect to BCRN stages, it should be further understood that all analysis is performed for each cancer type individually. As an example, a breast cancer observation data record cannot conflict with a rectal cancer observation data record. As illustrated in FIGS. 7A-7C, the pre-processing system executing the pre-processing methodology applies a plurality of filtering steps in sequence, such that a data record analyzed in accordance with any one of the described filtering steps has already been determined to satisfy all of the previously applied filtering steps. This sequential application of filtering steps enables complex filtering steps requiring simultaneous satisfaction of a plurality of factors to be applied in a simple manner.

As shown at Block 802, the BCRN stage pre-processing methodology, when executed by the pre-processing system, identifies observation data records having start dates within a range of dates identified as defining a relevant time frame for analysis. The date range may be defined within the configuration data for the pre-processing system in general, and may be defined in terms of a beginning date and an ending date for the date range (as shown at Block 803). Those records having dates that do not fall within the relevant date range are excluded from further analysis, as indicated at Block 811.

The BCRN pre-processing methodology executed by the pre-processing system continues by identifying data records having a concept type identifier indicating the observation data record relates to cancer stage, as indicated at Block 804. Those records that do not contain data elements indicating the observation data record relates to cancer stage data are excluded from further analysis, as indicated at Block 811.

The pre-processing system executing the BCRN pre-processing methodology continues by identifying data records indicating the data record source is from one or more permitted data sources, as indicated at Block 805. For example, the BCRN pre-processing methodology, when executed by the pre-processing system, may be configured to maintain observation data records obtained from a patient's EMR and/or from preauthorization request data (as indicated at Block 806). In certain embodiments, the BCRN pre-processing methodology is configured to cause the pre-processing system to accept data records from a single permitted data source. Those observation data records indicated as received from an unpermitted data source are excluded from further analysis, as indicated at Block 811.

The BCRN pre-processing methodology continues by causing the pre-processing system to identifying data records indicating the data record relates to breast cancer, colon cancer, rectal cancer, or NSCLC, as indicated at Block 807. Subsequent pre-processing methodologies implement BCRN-specific stage rules for identifying potentially conflicting or inconsistent data records (for a specific cancer type selected from breast cancer, colon cancer, rectal cancer, or NSCLC) and for identifying resolutions for identified data conflicts. Observation data records that do not relate to breast cancer, colon cancer, rectal cancer, or NSCLC are excluded from further analysis, as indicated at Block 811 (however such data records may be analyzed in accordance with other pre-processing methodologies as discussed herein if those data records are identified as relevant to those other pre-processing methodologies).

The BCRN pre-processing methodology is specifically configured to cause the pre-processing system to identify conflicts between data records based on differences in identified cancer stages. Accordingly, as indicated at Block 809, the BCRN pre-processing methodology causes the pre-processing system to determine whether an observation value identifier associated with each data record matches one of a defined number of available observation values relating to relevant cancer stage identifiers for BCRN. As shown at Block 810, which provides a list of relevant observation value identifiers, the BCRN pre-processing methodology causes the pre-processing system to determine whether a data record comprises an observation value identifier indicative of Stage 0 cancer, Stage I cancer, Stage II cancer, Stage III cancer, or Stage IV cancer. Those data records that do not contain observation value identifiers indicative of any of the acceptable observation value identifiers are omitted from BCRN stage processing, as indicated at Block 811.

After identifying records satisfying each of the filtering rules discussed in reference to Blocks 802-809, those records are retained for further analysis, as indicated at Block 812. Although not shown in FIGS. 7A-7C, in certain embodiments, the retained data records are modified by the addition of a derived observation value identifier field (and corresponding derived observation value identifier), which is populated with the observation value identifier provided as a part of the observation data record. By generating the derived observation value identifier field, the pre-processing system executing the BCRN pre-processing methodology may generate output data using the same file layout as pre-processed SCLC stage data, thus allowing all pre-processed stage output data to be output as one single file or multiple files with identical layouts, which in turn aids the processing of downstream analytics, such as machine-learning based severity models.

With reference specifically to FIG. 7B, for those records identified as having a concept condition identifier indicative of breast cancer, colon cancer, rectal cancer, or NSCLC, the BCRN pre-processing methodology continues by applying various filtering analyses by cancer type (as indicated at Block 813) for identifying and rectifying any identified conflicts between data records. The BCRN pre-processing methodology causes the pre-processing system to first identify intra-date conflicts, as discussed in reference to Blocks 814-826. Accordingly, as indicated at Block 814, the pre-processing system executing the BCRN pre-processing methodology identifies all observation records having a single start date to identify intra-day conflicts. If, according to the BCRN pre-processing methodology, the pre-processing system identifies only one observation record for a particular date (as shown at Block 815), the observation record is retained for further analysis, as indicated at Block 817. However, if more than one observation record is identified for a particular date, those identified records are further analyzed to determine whether they should be retained. As shown at Block 816, the BCRN pre-processing methodology then determines whether more than one observation value identifier is present for a particular day (or if the multiple observation data records all have the same observation value identifier). If only a single observation value identifier (only one stage value) is present for a given day, all of the observation data records for that date are retained for further analysis, as indicated at Block 817. However, if more than one observation value identifier is present for a given day, the BCRN pre-processing methodology proceeds, as indicated at Block 818, to determine whether any observation data records have an observation value identifier indicative of Stage IV cancer. If none of the observation data records have an observation value identifier indicative of Stage IV cancer (indicating that multiple different cancer stages are present on the same day and none of them are Stage IV), all of the records for that date are omitted from further BCRN stage processing, as indicated at Block 819.

However, if at least one Stage IV cancer observation value identifier is present on at least one observation data record, the intra-date conflict analysis of the BCRN pre-processing methodology continues, as indicated at Block 820, to retrieve relevant claims data, such that diagnosis codes may be utilized to address certain identified intra-date conflicts. The relevant claims data records are those claims data records having a first date of service on or before the first observation data record having a Stage IV cancer observation value identifier. Utilizing a lookup table mapping specific diagnosis codes to an indication of whether those diagnosis codes are indicative of a secondary malignancy (the lookup table reflected at Block 822), the BCRN pre-processing methodology causes the pre-processing system to determine whether any claims data records are indicative of a secondary malignancy, as indicated at Block 821. If no secondary malignancies are identified, then all observation data records for that particular date are excluded from further analysis, as indicated at Block 826. However, if a secondary malignancy is identified the observation data records for that date having a Stage IV cancer observation value identifier are retained for further processing (as indicated at Block 823) and any observation data records having Stage 0, Stage I, Stage, II, or Stage III observation value identifiers are excluded from further analysis, as indicated at Block 824 (with reference to a lookup table identifying observation value identifiers for each of these stages, as indicated at Block 825).

Although not specifically reflected within the flowchart of FIG. 7B, the BCRN pre-processing methodology may be further configured to cause the pre-processing system to implement one or more tiebreaker rules for eliminating entirely duplicative observation data records occurring on a single date. For example, tiebreaker rules may prioritize data generated by a particular data source, such that when duplicative data are available for a particular date, and one duplicative observation data record is generated by a preferred data source, the data record generated by the preferred data source is retained, and other observation data records are eliminated. As yet another example, a tiebreaker rule may prioritize data records having a lower unique record identifier (thereby prioritizing the “first” generated data record). Data records having higher unique record identifiers (generated after the first-generated data record) may be eliminated. As noted above, a plurality of tiebreaker rules may be implemented, such as in a hierarchical sequence, with subsequent tiebreaker rules being applied until all duplicative data records are eliminated.

FIG. 7C is a flowchart specifically focusing on subprocesses for identifying and rectifying inter-date conflicts identified between BRCN stage observation data records. As noted in Block 827, the process for identifying and rectifying inter-date conflicts identified between observation data records is performed for those observation data records that have not otherwise been excluded, for example, through the processes discussed in reference to FIGS. 7A-7B. As indicated at Block 828, the pre-processing system executing the BCRN pre-processing methodology determines whether there is more than one observation value identifier (more than one stage) associated with a particular patient for a particular time frame. If only one observation value identifier exists for the patient and time frame, the BCRN stage observation data record is written to an observation output file (as shown in block 829) to be accessed by downstream analytics including, but not limited to, computer-based modeling, such as machine-learning based severity models.

However, if more than one observation value identifier is identified, the BCRN pre-processing methodology causes the pre-processing system to determine whether any BCRN stage observation data records exist with a Stage IV observation value identifier, as indicated at Block 830. If the pre-processing system executing the BCRN pre-processing methodology determines that multiple BCRN stage observation records exist, but none of those multiple BCRN stage records have a Stage IV observation value identifier, all BCRN stage observation data records for all start dates are omitted from BCRN stage processing, as indicated at Block 831.

However, if a mixture of Stage IV observation data records and other stage observation data records (e.g., Stage 0, Stage I, Stage II, or Stage III) is identified for a particular patient and a particular time period, the inter-date conflict analysis continues, as reflected at Block 832, by determining a start date of the most-recent (latest date) BCRN stage observation data record having a Stage IV observation value identifier. Claims data records relevant to the patient and having a first date of service on or before the identified start date of the most-recent observation data record having a Stage IV observation value identifier are retrieved, as reflected at Block 833, and each retrieved claims data record is reviewed to determine whether any claims reflect a secondary malignancy diagnosis code as reflected at Block 834 (with reference to a lookup table correlating diagnosis codes with an indication of secondary malignancy, as reflected at Block 835).

If no secondary malignancy is identified with reference to the retrieved claims data, all observation data records for all start dates are omitted from BCRN stage processing, as reflected at Block 836. However, if at least one diagnosis code within a reviewed claims data record is indicative of a secondary malignancy, the observation data records having Stage IV BCRN observation value identifiers for all start dates are retained (as reflected at Block 837) and all observation data records having other BCRN observation value identifiers (Stage 0, Stage I, Stage II, or Stage III) for all start dates are omitted from BCRN stage processing, as reflected at Block 838 (with reference to a mapping data table identifying each of the excluded observation value identifiers, as reflected at Block 839).

The remaining retained data records after executing the BCRN pre-processing methodology are consistent for a particular patient, such that the retained data records do not indicate a changing BCRN stage for the particular patient either within a particular date, or across multiple dates within a relevant time period. Moreover, any conflicts identified (that could otherwise lead to inconsistent data for the patient) are rectified, for example, by eliminating data records for which a conflict cannot otherwise be eliminated, or by referencing additional data (e.g., claims data) to rectify certain conflicts, if appropriate. The remaining observation data records are written to an observation output file which can subsequently be used by a plurality of analytic processes including, but not limited to computer-based modeling. Moreover, it should be understood that all of the observation data records (including those excluded from further analysis) may be retained within a log data file for later auditing. As mentioned, the BCRN pre-processing methodology is repeated for all patients for whom patient identifiers are reflected within input data, before the BCRN pre-processing methodology ends, as reflected at Block 840 on FIG. 7A.

### Prostate Cancer

Next, the pre-processing system continues by implementing a prostate cancer pre-processing methodology. These multiple cancer-specific pre-processing methodologies may be executed in series or in parallel. As mentioned herein, the order in which the multiple pre-processing methodologies are executed may be changed, as the results of one pre-processing methodology are not dependent on any of the other pre-processing methodologies discussed herein (e.g., the prostate cancer pre-processing methodology is not dependent on the BCRN pre-processing methodology).

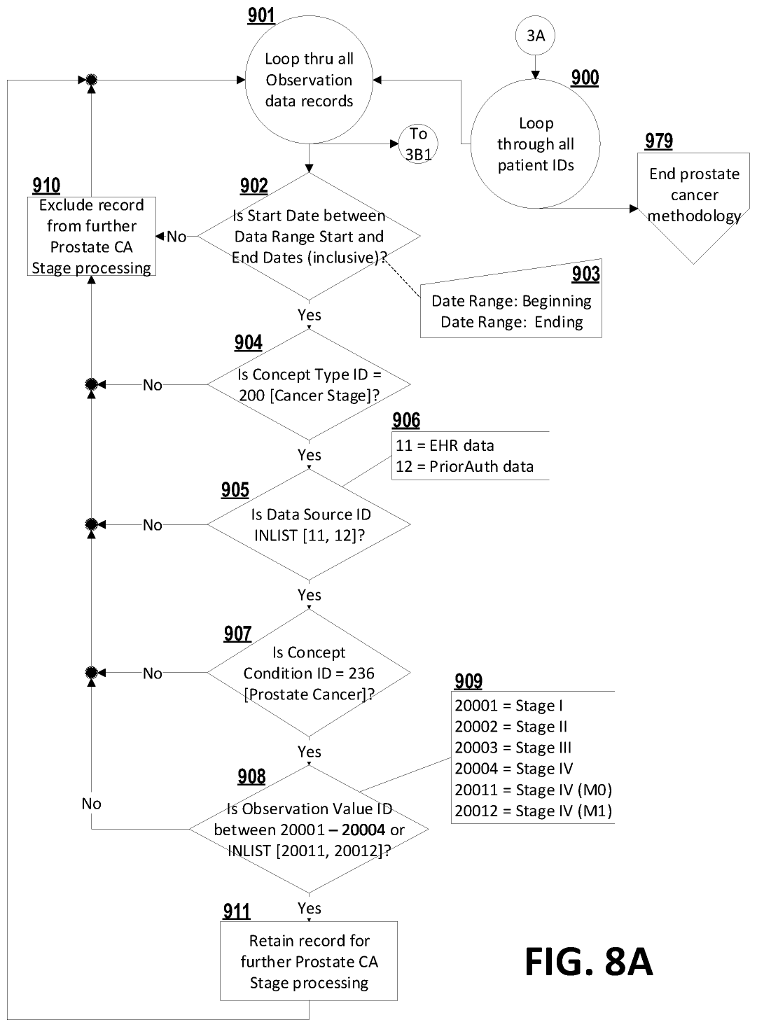

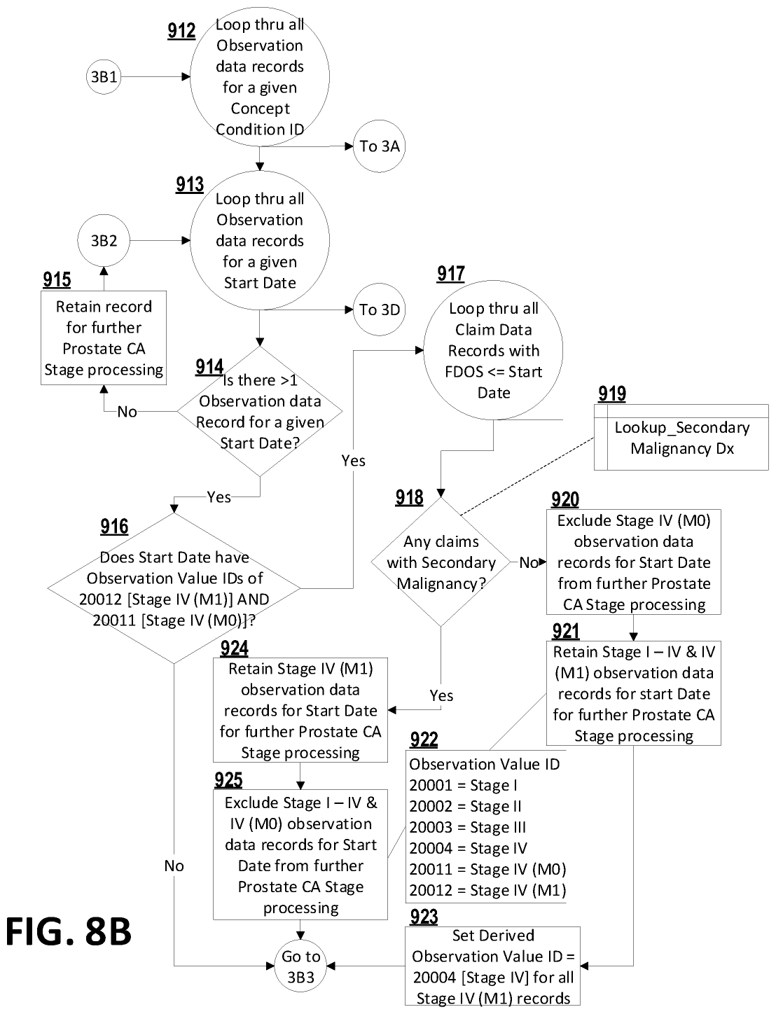

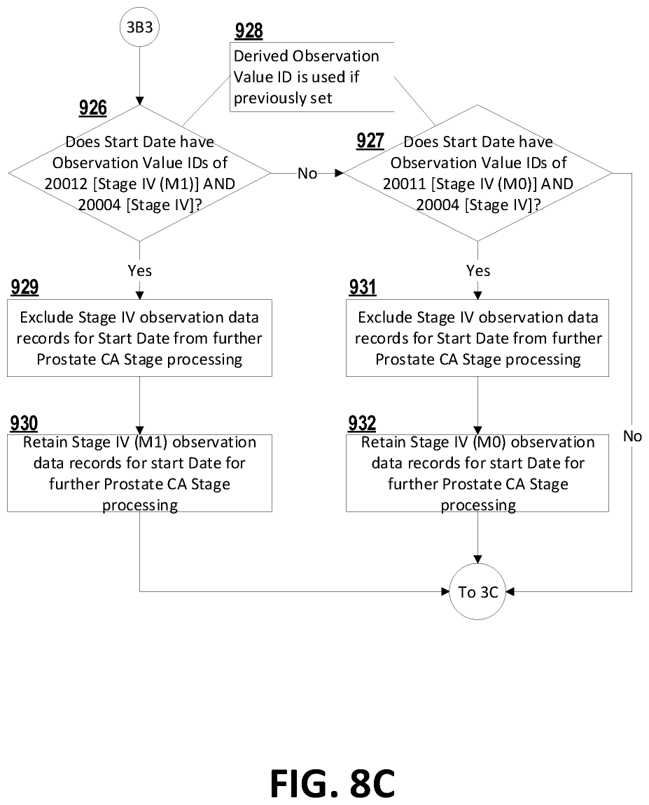

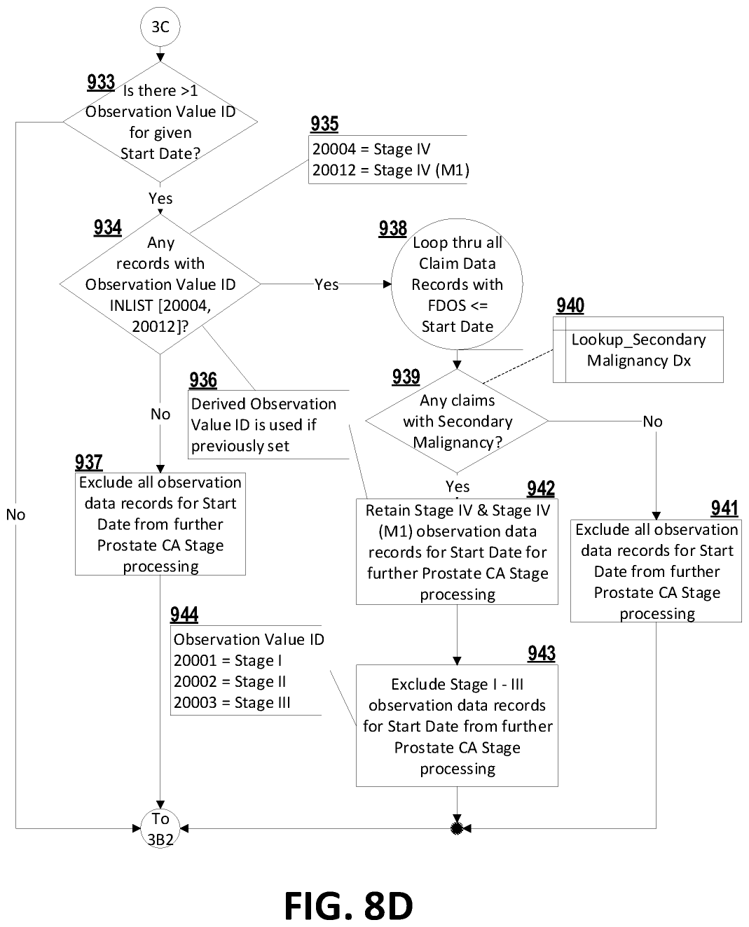

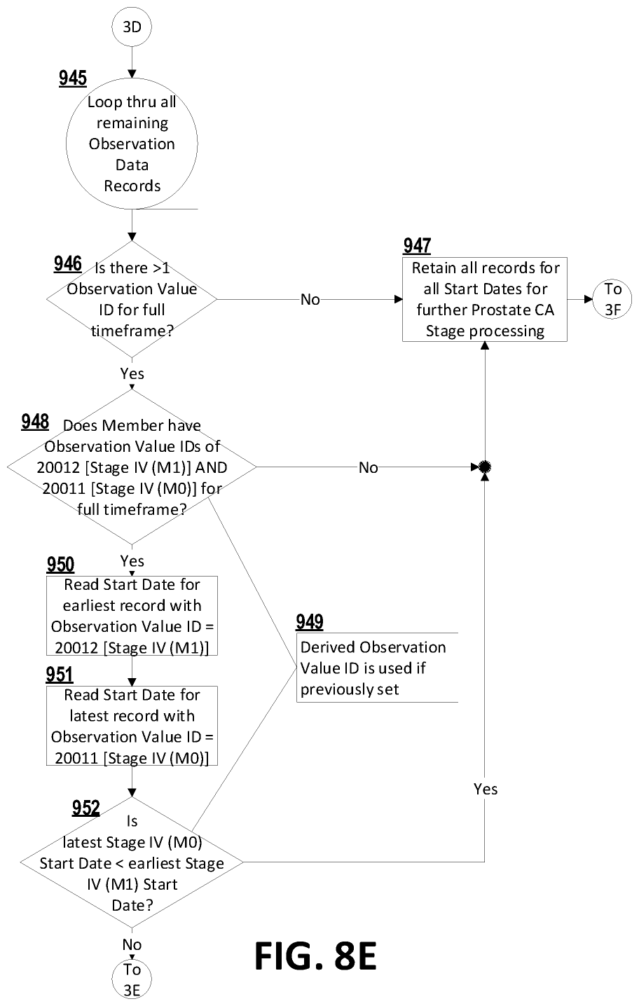

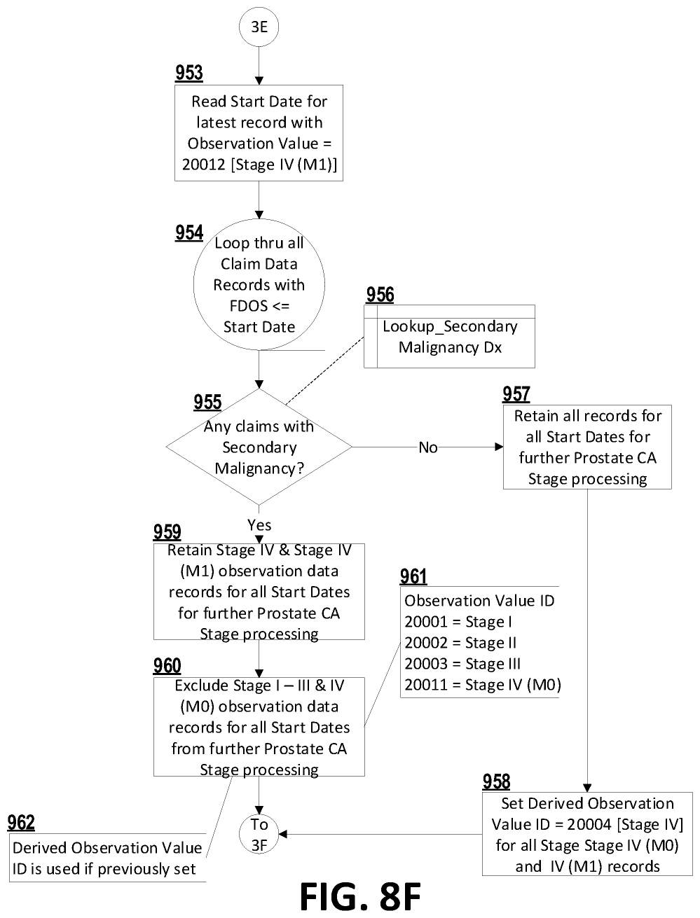

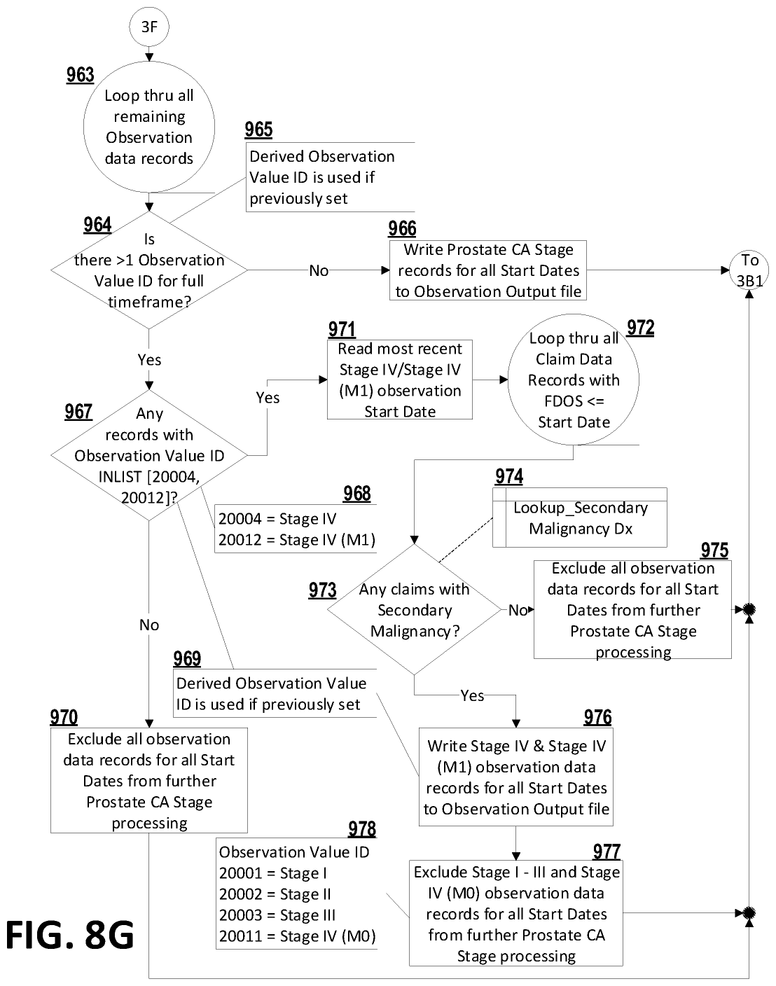

FIGS. 8A-8G collectively illustrate an example flowchart of a prostate cancer pre-processing methodology according to one embodiment. As shown at Block 900, the methodology reflected in the flowchart of FIGS. 8A-8G may be performed by the pre-processing system for all patients separately (the described methodology proceeding to analyze data records for a single patient before moving to a next patient). Moreover, the described steps reflected within FIGS. 8A-8G may be performed by looping through all records associated with the patient's unique identifier (as reflected within Block 901) so as to identify potentially conflicting and/or otherwise inconsistent data records or prostate cancer stage observations associated with the patient. As illustrated in FIGS. 8A-8G, the pre-processing system executing the pre-processing methodology applies a plurality of filtering steps in sequence, such that a data record analyzed in accordance with any one of the described filtering steps has already been determined to satisfy all of the previously applied filtering steps. This sequential application of filtering steps enables complex filtering steps requiring simultaneous satisfaction of a plurality of factors to be applied in a simple manner.

As shown in Block 902, the prostate cancer pre-processing methodology identifies observation data records having start dates within a range of dates identified as defining a relevant time frame for analysis. The date range may be defined within the configuration data for the pre-processing system in general and may be defined in terms of a beginning date and an ending date for the date range (as shown at Block 903). Those records having dates that do not fall within the relevant date range are excluded from further analysis, as indicated at Block 910.

The prostate cancer pre-processing methodology executed by the pre-processing system continues by identifying data records having a concept type identifier indicating the observation data record relates to cancer stage, as indicated at Block 904. Those records that do not contain data elements indicating the observation data record relates to cancer stage data are excluded from further analysis, as indicated at Block 910.

The prostate cancer pre-processing methodology continues by causing the pre-processing system to identify data records indicating the data record source is one of one or more permitted data sources, as indicated at Block 905. For example, the prostate cancer pre-processing methodology may be configured to cause the pre-processing system to maintain observation data records obtained from a patient's EMR and/or from preauthorization request data (as indicated at Block 906). In certain embodiments, the prostate cancer pre-processing methodology is configured to accept data records from a single permitted data source. Those observation data records indicated as received from an unpermitted data source are excluded from further analysis, as indicated at Block 910.

The pre-processing system executing the prostate cancer pre-processing methodology continues by identifying data records indicating the data record relates to prostate cancer, as indicated at Block 907. Subsequent pre-processing methodologies implement prostate cancer-specific stage rules for identifying potentially conflicting or inconsistent data records and for identifying resolutions for identified data conflicts when possible. Observation data records that do not relate to prostate cancer are excluded from further analysis as indicated at Block 910 (however such data records may be analyzed in accordance with other pre-processing methodologies as discussed herein if those data records are identified as relevant to those other pre-processing methodologies).

The prostate cancer pre-processing methodology is specifically configured to cause the pre-processing system to identify conflicts between data records based on differences in identified cancer stages for prostate cancer. Accordingly, as indicated at Block 908, the prostate cancer pre-processing methodology causes the pre-processing system to determine whether an observation value identifier associated with each data record matches one of a defined number of observation values relating to relevant cancer stage identifiers for prostate cancer. As shown at Block 909, which provides an example list of potential relevant observation value identifiers, the prostate cancer pre-processing methodology may determine whether a data record comprises an observation value identifier indicative of a Stage I cancer, Stage II cancer, Stage III cancer, Stage IV cancer, Stage IV (M0) cancer, or Stage IV (M1) cancer. Those data records that do not contain observation value identifiers indicative of any of the acceptable observation value identifiers are omitted from prostate cancer stage processing, as indicated at Block 910.

After identifying records satisfying each of the filtering rules discussed in reference to Blocks 902-908, those records are retained for further analysis, as indicated at Block 911. Although not shown in FIGS. 8A-8G, in certain embodiments, the retained data records are modified by the addition of a derived value identifier field (and corresponding derived observation value identifier), which is populated with the observation value identifier provided as a part of the observation data record. By generating the derived observation value identifier field, the prostate cancer pre-processing methodology causes the pre-processing system to utilize the same file layout as the pre-processed SCLC or BRCN stage data, thus allowing all pre-processed clinical stage data to output to one single file, or multiple files with identical layouts, which in turn aids the processing of downstream analytics.

With reference specifically to FIGS. 8B-8E, for those records identified as having a concept condition identifier indicative of prostate cancer, the prostate cancer pre-processing methodology continues by causing the pre-processing system to apply various filtering analyses (as indicated at Block 912) for identifying and rectifying any identified conflicts between data records. The pre-processing system executing the prostate cancer pre-processing methodology first identifies intra-date conflicts, as discussed in reference to Blocks 913-943 (FIGS. 8B-8D). Accordingly, as indicated at Block 913, the pre-processing system executing the prostate cancer pre-processing methodology identifies all observation records for a given start date to identify same-day conflicts. If, according to the prostate cancer pre-processing methodology, the pre-processing system identifies only one observation record for a particular date (as shown at Block 914), the observation record is retained for further analysis, as indicated at Block 915. However, if more than one observation record is identified for a particular date, those identified records are further analyzed to determine whether they should be retained. As shown at Block 916, the pre-processing system executing the prostate cancer pre-processing methodology determines whether there is at least one observation data record having a Stage IV (M1) observation value identifier and at least one observation data record having a Stage IV (M0) observation value identifier. If a particular start date does not have both a Stage IV (M1) observation value identifier and a Stage IV (M0) observation value identifier, then all records for the particular day are passed for further processing, as indicated at Block 926.

However, if a particular day has at least one observation data record having a Stage IV (M1) observation value identifier and at least one observation data record having a Stage IV (M0) observation value identifier, the intra-date conflict analysis of the prostate cancer pre-processing methodology continues as indicated at Block 917 by causing the pre-processing system to retrieve relevant claims data, such that diagnosis codes may be utilized to address certain identified intra-date conflicts. The relevant claims data records are those claims data records having a first date of service on or before the single date being analyzed. Utilizing a lookup table mapping specific diagnosis codes to an indication of whether those diagnosis codes are indicative of a secondary malignancy (the lookup table reflected at Block 919), the prostate cancer pre-processing methodology causes the pre-processing system to determine whether any claims data records are indicative of a secondary malignancy, as indicated at Block 918. If no secondary malignancies are identified, then the observation data records for that date having a Stage IV (M0) observation value identifier are excluded from further analysis, as indicated at Block 920, and other observation data records for that date (those having an observation value identifier of Stage I, Stage II, Stage III, Stage IV, or Stage IV (M1)) are retained for further analysis, as indicated at Block 921 (with reference to a lookup table identifying those observation value identifiers, as indicated at Block 922). For those observation data records retained for further analysis and indicative of an observation value identifier of Stage IV (M1), a derived observation value identifier for each of the retained observation data records within the model input data set is set to a Stage IV indicator for further analysis, as indicated at Block 923. Other retained observation data records may set a derived observation value identifier to be equal to the original observation value identifier, to facilitate further processing (such that a single data field may be utilized during subsequent analytics).

However, if a secondary malignancy is identified within the analyzed claims data, the observation data records for that date having a Stage IV (M1) observation value identifier are retained for further analysis, as indicated at Block 924, and all other observation data records for that date (those having an observation value identifier of Stage I, Stage II, Stage III, Stage IV, or Stage IV (M0)) are excluded from further analysis, as indicated at Block 925 (with reference to the lookup table identifying those observation value identifiers, as indicated at Block 922).

FIG. 8C is a flowchart providing additional steps of processing intra-date conflicts. For those records retained after processing according to FIGS. 8A-B, the pre-processing system executing the prostate cancer pre-processing methodology provides additional analysis to determine whether there are any conflicts in Stage IV records remaining. Specifically, as indicated in Blocks 926-927, the prostate cancer pre-processing methodology causes the pre-processing system to determine whether the retained observation data records include combinations of observation data records including Stage IV and Stage IV (M1) observation value identifiers (as indicated at Block 926) or combinations of observation data records including Stage IV and Stage IV (M0) observation value identifiers (as indicated at Block 927), with reference to derived observation value identifiers if generated in accordance with prior pre-processing steps, as indicated at Block 928. These determinations may be performed in sequence, as reflected in FIG. 8C (e.g., determining whether a combination of Stage IV and Stage IV (M1) observation value identifiers exist, and then determining whether a combination of Stage IV and Stage IV (M0) observation value identifiers exist if there is no combination of Stage IV and Stage IV (M1) observation value identifiers).

Upon identifying a combination of Stage IV and Stage IV (M1) observation value identifiers (as indicated at Block 926), the pre-processing system executing the prostate cancer pre-processing methodology excludes the Stage IV observation data records for that date from further processing (as indicated at Block 929) and retains the more specific Stage IV (M1) observation data records for that date for further processing (as indicated at Block 930).

Upon identifying a combination of Stage IV and Stage IV (M0) observation value identifiers (as indicated at Block 927), the prostate cancer pre-processing methodology causes the pre-processing system to exclude the Stage IV observation data records for that date from further processing (as indicated at Block 931) and retains the more specific Stage IV (M0) observation data records for that date for further processing (as indicated at Block 932).

The prostate cancer pre-processing methodology causes the pre-processing system to continue analysis of potential intra-date conflicts as indicated in FIG. 8D for those observation data records that are retained after pre-processing according to the subprocesses discussed in reference to FIGS. 8A-8C. As indicated at Block 933, the pre-processing system executing the prostate cancer pre-processing methodology determines whether there is more than one observation value identifier for a particular date. If only one observation value identifier is present for a particular date, the observation data records are retained for further analysis. However, if more than one observation value identifier is identified for a particular date, the pre-processing system executing the prostate cancer pre-processing methodology then causes the pre-processing system to determine whether any of the included observation value identifiers are indicative of Stage IV or Stage IV (M1) cancer stages, as indicated at Block 934 (with reference to a lookup table identifying the relevant observation value identifiers, as reflected at Block 935, and with reference to derived observation value identifiers, if relevant, as indicated at Block 936). If no observation value identifiers indicative of Stage IV or Stage IV (M1) cancer stages are present, then all observation records for the particular date are excluded from further analysis, as indicated at Block 937.

However, if at least one observation value identifier indicative of either Stage IV cancer or Stage IV (M1) cancer is identified, the intra-date conflict analysis of the prostate cancer pre-processing methodology causes the pre-processing system to continue as indicated at Block 938 to retrieve relevant claims data, such that diagnosis codes may be utilized to address certain identified intra-date conflicts. The relevant claims data records are those claims data records having a first date of service on or before the observation date being analyzed. Utilizing a lookup table mapping specific diagnosis codes to an indication of whether those diagnosis codes are indicative of a secondary malignancy (the lookup table reflected at Block 940), the pre-processing system executing the prostate cancer pre-processing methodology determines whether any claims data records are indicative of a secondary malignancy, as indicated at Block 939. If no secondary malignancies are identified, then all observation data records for the particular date are excluded from further analysis, as indicated at Block 941. However, if a secondary malignancy is identified, the observation data records for that date having a Stage IV or Stage IV (M1) observation value identifier are retained for further processing (as indicated at Block 942) and any observation data records for that date having a Stage I, Stage II, or Stage III observation value identifier are excluded from further analysis, as indicated at Block 943 (with reference to a lookup table identifying observation value identifiers for each of these stages, as indicated at Block 944).

FIGS. 8E-8F provide a flowchart specifically focusing on subprocesses for identifying and rectifying inter-date conflicts identified between observation data records having Stage IV observation value identifiers. As noted in Block 945, the process for identifying and rectifying inter-date conflicts identified between observation data records containing Stage IV observation value identifiers is performed for those observation data records that have not otherwise been excluded, for example, through the processes discussed in reference to FIGS. 8A-8D. As indicated at Block 946, the prostate cancer pre-processing methodology causes the pre-processing system to determine whether there is more than one observation value identifier (more than one stage value) associated with a particular patient for a particular time frame. If only one observation value identifier exists for the patient and time frame, the prostate cancer stage observation record is retained for further processing, as indicated at Block 947.

However, if more than one observation value identifier is identified, the prostate cancer pre-processing methodology continues when executed by the pre-processing system to determine whether the member has at least one Stage IV (M1) observation value identifier and at least one Stage IV (M0) observation value identifier, as indicated at Block 948. The prostate cancer pre-processing methodology may reference derived observation value identifiers, if relevant, for analyzed observation data records, as indicated by block 949. If the member does not have at least one Stage IV (M1) observation value identifier and at least one Stage IV (M0) observation value identifier, the prostate cancer stage observation records are retained for further processing, as indicated at Block 947.

If the prostate cancer pre-processing methodology results in a determination that the member has at least one Stage IV (M1) observation value identifier and at least one Stage IV (M0) observation value identifier, the prostate cancer pre-processing methodology causes the pre-processing system to determine the start date for the earliest observation data record having a Stage IV (M1) observation value identifier (as indicated at Block 950) and a start date for the latest observation data record having a Stage IV (M0) observation value identifier (as indicated at Block 951), thereby enabling a determination of whether the latest Stage IV (M0) observation data record occurs before the earliest Stage IV (M1) observation data record, as reflected at Block 952. If the latest Stage IV (M0) observation data record occurs before the first Stage IV (M1) observation data record, all of the observation data records are retained for further analysis, as indicated at Block 947.

However, if the latest Stage IV (M0) observation data record occurs after the first Stage IV (M1) observation data record, the prostate cancer pre-processing methodology proceeds to Block 953 to cause the pre-processing system to retrieve relevant claims data, such that diagnosis codes may be utilized to address certain identified inter-date conflicts. Specifically, as indicated at Block 953, the pre-processing system executing the prostate cancer pre-processing methodology identifies a start date for the latest Stage IV (M1) observation data record, and then identifies claims data records for the member having a first date of service occurring on or before the identified start date for the latest Stage IV (M1) observation data record, as indicated at Block 954. Utilizing a lookup table mapping specific diagnosis codes to an indication of whether those diagnosis codes are indicative of a secondary malignancy (the lookup table reflected at Block 956), the prostate cancer pre-processing methodology, when executed by the pre-processing system, determines whether any claims data records are indicative of a secondary malignancy, as indicated at Block 955. If no secondary malignancies are identified, then all observation data records are retained for further analysis as indicated at Block 957. For those retained observation data records, the pre-processing system executing the prostate cancer pre-processing methodology sets a derived observation value identifier to reflect a Stage IV observation value identifier for all those observation data records having a Stage IV (M0) or Stage IV (M1) original observation value identifier, as indicated at Block 958.

However if a secondary malignancy is identified, the observation data records having Stage IV observation value identifiers or Stage IV (M1) observation value identifiers are retained for further analysis as indicated at Block 959, and observation data records having Stage I, Stage II, Stage III, or Stage IV (M0) observation value identifiers are omitted from prostate cancer stage processing, as indicated at Block 960 (with reference to a lookup table identifying observation value identifiers for each of these stages, as indicated at Block 961 and with reference to a derived observation value identifier, if relevant and as indicated at Block 962).

FIG. 8G is a flowchart specifically focusing on subprocesses for identifying and rectifying inter-date conflicts identified between observation data records for all prostate cancer stages. As noted in Block 963, the process for identifying and rectifying inter-date conflicts identified between observation data records is performed for those observation data records that have not otherwise been excluded, for example, through the processes discussed in reference to FIGS. 8A-8F. As indicated at Block 964, the prostate cancer pre-processing methodology causes the pre-processing system to determine whether there is more than one observation value identifier associated with a particular patient for a particular time frame (with reference to derived observation value identifiers, if relevant, as indicated at Block 965). If only one observation value identifier exists for the patient and time frame, the pre-processing system executing the prostate cancer pre-processing methodology retains the prostate cancer observation data record as reflected at Block 966.

However, if more than one observation value identifier is identified, the pre-processing system executing the prostate cancer pre-processing methodology determines whether observation data records exist with a Stage IV observation value identifier or a Stage IV (M1) observation value identifier, as reflected at Block 967 (with reference to a lookup table, as indicated at Block 968, and derived observation value identifiers, if relevant and as indicated at Block 969). If the prostate cancer pre-processing methodology causes the pre-processing system to determine that multiple prostate cancer observation data records exist, but none of those multiple prostate cancer observation data records have a Stage IV observation value identifier or a Stage IV (M1) observation value identifier, all prostate cancer observation data records for all start dates are excluded from further processing, as indicated at Block 970.

If, at Block 967, the prostate cancer pre-processing methodology results in a determination that there is at least one observation data record having a Stage IV observation value identifier or a Stage IV (M1) observation value identifier, the inter-date conflict analysis continues, as reflected at Block 971, by determining a start date of the most recent (latest date) prostate cancer observation data record having a Stage IV observation value identifier or a Stage IV (M1) observation value identifier. Claims data records relevant to the patient and having a first date of service on or before the identified start date of the most recent observation data record having a Stage IV observation value identifier or a Stage IV (M1) observation value identifier are retrieved, as reflected at Block 972, and each retrieved claims data record is reviewed to determine whether any claims reflect a secondary malignancy diagnosis code as reflected at Block 973 (with reference to a lookup table correlating diagnosis codes with an indication of secondary malignancy, as reflected at Block 974).

If no secondary malignancy is identified with reference to the retrieved claims data, all observation data records for all start dates are omitted from prostate cancer stage processing, as reflected at Block 975. However, if at least one diagnosis code within a reviewed claims data record is indicative of a secondary malignancy, the observation data records having Stage IV and Stage IV (M1) observation value identifiers for all start dates are retained (and as reflected at Block 976) and all observation data records having other stage observation value identifiers (Stage I, Stage II, Stage III, and Stage IV (M0) for all start dates are omitted from prostate cancer stage processing, as reflected at Block 977 (with reference to a mapping data table listing identifiers for each of the excluded observation value identifiers, as reflected at Block 978).

As mentioned, the prostate cancer pre-processing methodology is repeated for all patients for whom patient identifiers are reflected within input data, before the prostate cancer pre-processing methodology ends, as reflected at Block 979 on FIG. 8A.

Although not specifically reflected within the flowchart of FIGS. 8A-8G, the prostate cancer pre-processing methodology may be further configured to cause the pre-processing system to implement one or more tiebreaker rules for eliminating entirely duplicative observation data records occurring on a single date. For example, tiebreaker rules may prioritize data generated by a particular data source, such that when duplicative data are available for a particular date, and one duplicative observation data record is generated by the a preferred data source, the data record generated by the preferred data source is retained, and other observation data records are eliminated. As yet another example, a tiebreaker rule may prioritize data records having a lower unique record identifier (thereby prioritizing the “first” generated data record). Data records having higher unique record identifiers (generated after the first-generated data record) may be eliminated. As mentioned above, a plurality of tiebreaker rules may be implemented in certain embodiments, such as in a hierarchical sequence. The plurality of tiebreaker rules may be sequentially applied in accordance with the hierarchy until all duplicative observation data records are eliminated.

The remaining retained data records in the output data set after the pre-processing system executes the prostate cancer stage pre-processing methodology are consistent for a particular patient, such that the retained data records within the output data set do not indicate a changing prostate cancer stage for the particular patient either within a particular date, or across multiple dates within a relevant time period. Moreover, any conflicts identified (that could otherwise lead to inconsistent data for the patient) are rectified, for example, by eliminating data records for which a conflict cannot otherwise be eliminated, or by referencing additional data (e.g., claims data) to rectify certain conflicts, if appropriate. Moreover, it should be understood that all of the observation data records (including those excluded from further analysis) may be retained within a log data file for later auditing.

### Data Output

The pre-processing processes discussed above generate one or more model input data sets (as output of the pre-processing processes) that are free from internal data inconsistences between included observation data records, such as inconsistencies between observation data records generated on a single date and/or inconsistencies between observation data records generated over a multi-day time period (e.g., one month, one year, and/or the like).

Each model input data set includes a plurality of observation data records. Each observation data record comprises a plurality of data elements, and each observation data record may additionally comprise one or more metadata elements associated therewith.

The observation data record may comprise data elements indicative of a first start date associated with the record, a data source identifier, a concept type identifier (e.g., identifying the record as providing cancer stage data, such as via proprietary codes of an ontology), a concept condition type (cancer type identifier which may be a proprietary code of an ontology), an observation value identifier (e.g., indicative of a cancer stage, as provided as input for the observation data record), a derived observation value identifier (e.g., indicative of a cancer stage, as generated by the pre-processing system and methodology, which may match the observation value identifier or may differ from the observation value identifier), and/or the like.

As mentioned, each observation data record of the data output may be associated with metadata providing additional data regarding the observation data record. The metadata may comprise a unique patient identifier such that the data record may be correlated with a particular patient, a unique record identifier such that an individual data record may be separately identified, a data source identifier, a log code (which may indicate a reason why the corresponding data record was excluded from the output, if applicable), and/or the like.

In certain embodiments, the data output may be subject to one or more limitations that are executed in accordance with various tie breaker rules. For example, the data output may be subject to a limitation that only a single output observation data record may be included within a model input data set for a given observation date. As mentioned above, the tiebreaker rules may be configured to eliminate duplicative observation data records for a given observation start date, such as by retaining an observation data record having a lower unique record identifier (selected between duplicative observation data records). Other tiebreaker rules may implement a preferred hierarchy of data sources, such that observation data records received from a more highly preferred data source (e.g., which may be indicated based on a lookup table storing the hierarchy of preferred data sources) are retained over duplicative data records received from lower ranked data sources. In such embodiments, the observation data records received from the lower ranked data sources are excluded from further analysis. Data records which are excluded from further analysis may be stored within a log data file for later auditing.

### Severity Modelling

The output data set (also recited herein as model input data set from the output of the pre-processing methodologies), including consistent data records for a particular cancer type having a consistent indication of cancer stage for the cancer type of a particular patient, may then be provided to one or more severity models (and/or other analytics) that may be utilized for generating severity data, which may be indicative of the patient's cancer (and/or other data, as generated according to an alternative down-stream analytic), thereby providing a higher level of granularity regarding a patient's characteristics, such as the cost of treating the patient's cancer. Collectively, the pre-processing methodology and the severity models define a method for intaking potentially inconsistent observation data records, rectifying inconsistencies and providing the observation data records for severity modeling, which may generate data indicative of expected costs related to treatment of a patient's cancer. The data generated via the severity modeling may additionally reflect other aspects of a patient's health beyond the patient's cancer stage indication, which may have an impact on the complexity (and costs) associated with treating the patient's cancer. For example, the severity models may generate severity data based at least in part on data indicative of the patient's age and gender, data indicative of comorbidities of the patient (e.g., encompassing other medical conditions that may complicate treatment of a particular cancer), and additional data indicative of the patient's condition status as relates to their cancer diagnosis. In certain embodiments, the severity score generated by the severity model may be accompanied by one or more severity attributes indicative of an estimated cost for treating the patient's cancer.

The severity models may be implemented by the management computing entity 10, and/or another computing entity. The severity models may be configured to generate a severity score that may be utilized to compare the relative expected treatment cost of a patient's cancer. The severity score may have no associated units (such that the severity score is simply a number that can be compared against other generated severity scores). In other embodiments, the severity score may have an associated unit, such as a cost (the units may be embodied as US dollars) that may be reflective of a predicted cancer treatment cost associated with treating the patient's cancer. For example, a severity score may be reflective of a predicted medically necessary cancer treatment cost for a patient's cancer, considering the specific circumstances of the particular patient's condition. It should be understood that other measurement units may be associated with the severity score in certain embodiments.

The severity models (and/or other models provided to generate other data relating to a patient's cancer diagnosis) may be embodied as machine-learning based models, such as regularized linear regression models (e.g., Least Absolute Shrinkage and Selection Operator (LASSO) linear regression models), logistic regression models, neural networks, random forest models, clustering models, and/or the like, that may utilize a training data set to self-develop models for generating appropriate severity models (or other models) for various cancer types. It should be understood that a single severity model may be utilized for all cancer types, or a plurality of severity models may be implemented, with each severity model corresponding to a single cancer type or other subset of model input data set types.

In certain embodiments, a training data set may be generated utilizing retrospective model input data sets generated via the above-mentioned pre-processing configurations, combined with additional data, such as data indicative of corresponding costs that resulted from treatment of the patient's cancer. Thus, the training data sets include data reflecting inputs to the severity models as well as data reflecting an actual result corresponding with treatment of the patient's cancer—the actual result may be utilized as the dependent variable when training the model. Accordingly, the training data set may be provided for machine learning of the severity model (e.g., supervised or unsupervised), thereby enabling automated generation of severity models for use in analyzing model input data sets resulting from the pre-processing configurations discussed above.

### CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A computer-implemented method for automatically modeling severity attributes of a cancer treatment utilizing a plurality of independently generated observation data records for a patient, the method comprising:
receiving a plurality of independently generated observation data records each comprising structured observation data for a patient;
identifying a condition-specific plurality of observation data records selected from the plurality of independently generated observation data records, wherein the condition-specific plurality of observation data records are embodied as observation data records all comprising a common cancer type identifier;
filtering the plurality of condition-specific observation data records to eliminate one or more data records failing to satisfy one or more preliminary filter criteria;
based at least in part on the common cancer type identifier, initiating a pre-processing process for the condition-specific observation data records, wherein the pre-processing process sequentially executes a plurality of subprocesses to exclude one or more data records and to generate one or more output data records for the condition-specific observation data records having at least one shared identifier;
providing the one or more output data records to a selected machine-learning based model selected from a plurality of machine-learning based models based at least in part on one or more identifiers present within at least one of the one or more output data records; and
generating, via the selected machine-learning based model, a severity score indicative of one or more severity attributes for the patient.

2. The computer-implemented method of claim 1, wherein initiating a pre-processing process further comprises:
selecting a pre-processing process for the condition-specific observation data records from a plurality of available pre-processing processes comprising:
a first available pre-processing process applicable to one or more first cancer type identifiers, wherein the first available pre-processing process is configured to output a model input data set comprising one or more data records comprising a relevant cancer stage identifier selected from a first plurality of cancer stage identifiers applicable to the one or more first cancer type identifiers;
a second available pre-processing process applicable to one or more second cancer type identifiers, wherein the second available pre-processing process is configured to output a model input data set comprising one or more data records comprising a relevant cancer stage identifier selected from a second plurality of cancer stage identifiers applicable to the one or more second cancer type identifiers; and
a third available pre-processing process applicable to one or more third cancer type identifiers, wherein the third available pre-processing process is configured to output a model input data set comprising one or more data records comprising a relevant cancer stage identifier selected from a third plurality of cancer stage identifiers applicable to the one or more third cancer type identifiers.

3. The computer-implemented method of claim 2, wherein:
the one or more first cancer type identifiers comprise a Small-Cell Lung Cancer (SCLC) identifier, and the first available pre-processing process is configured to output a model input data set comprising one or more data records comprising a relevant cancer stage identifier selected from a limited stage identifier or an extensive stage identifier;
the one or more second cancer type identifiers comprise one or more of: (a) a breast cancer identifier, (b) a colon cancer identifier, (c) a rectal cancer identifier, or (d) a Non-Small-Cell Lung Cancer (NSCLC) identifier; and the second available pre-processing process is configured to output a model input data set comprising one or more data records comprising a relevant cancer stage identifier selected from: a Stage 0 identifier, a Stage I identifier, a Stage II identifier, a Stage III identifier, or a Stage IV identifier; and
the one or more third cancer type identifiers comprise a prostate cancer identifier, and the third available pre-processing process is configured to output a model input data set comprising one or more data records comprising a relevant cancer stage identifier selected from: a Stage I identifier, a Stage II identifier, a Stage III identifier, a Stage IV identifier, a Stage IV(M0) identifier, or a Stage IV(M1) identifier.

4. The computer-implemented method of claim 1, wherein the preliminary filter criteria comprise one or more of:
a date-based filter criterion for selecting independently generated observation data records for further analysis as generated within a defined date range;
a data source filter criterion for selecting independently generated observation data records for further analysis as generated by one or more defined data sources; or
a data content filter criterion for selecting independently generated observation data records for further analysis as containing an identifier selected from a plurality of available identifiers eligible for further analysis.

5. The computer-implemented method of claim 1, wherein the machine learning based model is a linear regression model.

6. The computer-implemented method of claim 1, wherein the sequentially executed plurality of subprocesses are configured to:
exclude one or more observation data records identified as failing to satisfy a rule of a subprocess for an intra-date conflict between cancer stage identifiers within observation data records having a common date; and
exclude one or more observation data records identified as failing to satisfy a rule of a subprocess for an inter-date conflict between cancer stage identifiers within observation data records having different dates.

7. The computer-implemented method of claim 1, wherein at least one of the sequentially executed plurality of subprocesses is configured to retrieve one or more claims data records comprising diagnostic data to identify at least one observation data record to retain within the model input data set.

8. The computer-implemented method of claim 7, wherein the at least one of the sequentially executed plurality of subprocesses is further configured to generate a derived data element within an observation data record included within the model input data set based at least in part on the claims data records.

9. A system comprising one or more memory storage areas and one or more processors for automatically modeling severity attributes of a cancer treatment utilizing a plurality of independently generated observation data records for a patient, the one or more processors are collectively configured to:
receive a plurality of independently generated observation data records each comprising structured observation data for a patient;
identify a condition-specific plurality of observation data records selected from the plurality of independently generated observation data records, wherein the condition-specific plurality of observation data records are embodied as observation data records all comprising a common cancer type identifier;
filter the plurality of condition-specific observation data records to eliminate one or more condition-specific observation data records failing to satisfy one or more preliminary filter criteria;
based at least in part on the common cancer type identifier, initiate a pre-processing process for the condition-specific observation data records, wherein the pre-processing process sequentially executes a plurality of subprocesses to exclude one or more condition-specific observation data records and to generate one or more output data records for the condition-specific observation data records having at least one shared identifier;
providing the one or more output data records to a selected machine-learning based model selected from a plurality of machine-learning based models based at least in part on one or more identifiers present within at least one of the one or more output data records; and
generating, via the selected machine-learning based model, a severity score indicative of one or more severity attributes for the patient.

10. The system of claim 9, wherein initiating a pre-processing process further comprises:
selecting a pre-processing process for the condition-specific observation data records from a plurality of available pre-processing processes comprising:
a first available pre-processing process applicable to one or more first cancer type identifiers, wherein the first available pre-processing process is configured to output a model input data set comprising one or more data records comprising a relevant cancer stage identifier selected from a first plurality of cancer stage identifiers applicable to the one or more first cancer type identifiers;
a second available pre-processing process applicable to one or more second cancer type identifiers, wherein the second available pre-processing process is configured to output a model input data set comprising one or more data records comprising a relevant cancer stage identifier selected from a second plurality of cancer stage identifiers applicable to the one or more second cancer type identifiers; and
a third available pre-processing process applicable to one or more third cancer type identifiers, wherein the third available pre-processing process is configured to output a model input data set comprising one or more data records comprising a relevant cancer stage identifier selected from a third plurality of cancer stage identifiers applicable to the one or more third cancer type identifiers.

11. The system of claim 10, wherein:
the one or more first cancer type identifiers comprise a Small-Cell Lung Cancer (SCLC) identifier, and the first available pre-processing process is configured to output a model input data set comprising one or more data records comprising a relevant cancer stage identifier selected from a limited stage identifier or an extensive stage identifier;
the one or more second cancer type identifiers comprise one or more of: (a) a breast cancer identifier, (b) a colon cancer identifier, (c) a rectal cancer identifier, or (d) a Non-Small-Cell Lung Cancer (NSCLC) identifier; and the second available pre-processing process is configured to output a model input data set comprising one or more data records comprising a relevant cancer stage identifier selected from: a Stage 0 identifier, a Stage I identifier, a Stage II identifier, a Stage III identifier, or a Stage IV identifier; and
the one or more third cancer type identifiers comprise a prostate cancer identifier, and the third available pre-processing process is configured to output a model input data set comprising one or more data records comprising a relevant cancer stage identifier selected from: a Stage I identifier, a Stage II identifier, a Stage III identifier, a Stage IV identifier, a Stage IV(M0) identifier, or a Stage IV(M1) identifier.

12. The system of claim 9, wherein the preliminary filter criteria comprise one or more of:
a date-based filter criterion for selecting independently generated observation data records for further analysis as generated within a defined date range;
a data source filter criterion for selecting independently generated observation data records for further analysis as generated by one or more defined data sources; or
a data content filter criterion for selecting independently generated observation data records for further analysis as containing an identifier selected from a plurality of available identifiers eligible for further analysis.

13. The system of claim 9, wherein the machine learning based model is a linear regression model.

14. The system of claim 9, wherein the sequentially executed plurality of subprocesses are configured to:
exclude one or more observation data records identified as failing to satisfy a rule of a subprocess for an intra-date conflict between cancer stage identifiers within observation data records having a common date; and
exclude one or more observation data records identified as failing to satisfy a rule of a subprocess for an intra-date conflict between cancer stage identifiers within observation data records having different dates.

15. A computer program product for automatically modeling severity attributes of a cancer treatment utilizing a plurality of independently generated observation data records for a patient, the computer program product comprising at least one non-transitory computer-readable storage medium having computer-readable program code portions stored therein, the computer-readable program code portions configured to:
receive a plurality of independently generated observation data records each comprising structured observation data for a patient;
identify a condition-specific plurality of observation data records selected from the plurality of independently generated observation data records, wherein the condition-specific plurality of observation data records are embodied as observation data records all comprising a common cancer type identifier;
filter the plurality of condition-specific observation data records to eliminate one or more condition-specific observation data records failing to satisfy one or more preliminary filter criteria;
based at least in part on the common cancer type identifier, initiate a pre-processing process for the condition-specific observation data records, wherein the pre-processing process sequentially executes a plurality of subprocesses to exclude one or more condition-specific observation data records and to generate one or more output data records for the condition-specific observation data records having at least one shared identifier;
providing the one or more output data records to a selected machine-learning based model selected from a plurality of machine-learning based models based at least in part on one or more identifiers present within at least one of the one or more output data records; and
generating, via the selected machine-learning based model, a severity score indicative of one or more severity attributes for the patient.

16. The computer program product of claim 15, wherein initiating a pre-processing process further comprises:
selecting a pre-processing process for the condition-specific observation data records from a plurality of available pre-processing processes comprising:
a first available pre-processing process applicable to one or more first cancer type identifiers, wherein the first available pre-processing process is configured to output a model input data set comprising one or more data records comprising a relevant cancer stage identifier selected from a first plurality of cancer stage identifiers applicable to the one or more first cancer type identifiers;
a second available pre-processing process applicable to one or more second cancer type identifiers, wherein the second available pre-processing process is configured to output a model input data set comprising one or more data records comprising a relevant cancer stage identifier selected from a second plurality of cancer stage identifiers applicable to the one or more second cancer type identifiers; and
a third available pre-processing process applicable to one or more third cancer type identifiers, wherein the third available pre-processing process is configured to output a model input data set comprising one or more data records comprising a relevant cancer stage identifier selected from a third plurality of cancer stage identifiers applicable to the one or more third cancer type identifiers.

17. The computer program product of claim 16, wherein:
the one or more first cancer type identifiers comprise a Small-Cell Lung Cancer (SCLC) identifier, and the first available pre-processing process is configured to output a model input data set comprising one or more data records comprising a relevant cancer stage identifier selected from a limited stage identifier or an extensive stage identifier;
the one or more second cancer type identifiers comprise one or more of: (a) a breast cancer identifier, (b) a colon cancer identifier, (c) a rectal cancer identifier, or (d) a Non-Small-Cell Lung Cancer (NSCLC) identifier; and the second available pre-processing process is configured to output a model input data set comprising one or more data records comprising a relevant cancer stage identifier selected from: a Stage 0 identifier, a Stage I identifier, a Stage II identifier, a Stage III identifier, or a Stage IV identifier; and
the one or more third cancer type identifiers comprise a prostate cancer identifier, and the third available pre-processing process is configured to output a model input data set comprising one or more data records comprising a relevant cancer stage identifier selected from: a Stage I identifier, a Stage II identifier, a Stage III identifier, a Stage IV identifier, a Stage IV(M0) identifier, or a Stage IV(M1) identifier.

18. The computer program product of claim 15, wherein the preliminary filter criteria comprise one or more of:
a date-based filter criterion for selecting independently generated observation data records for further analysis as generated within a defined date range;
a data source filter criterion for selecting independently generated observation data records for further analysis as generated by one or more defined data sources; or
a data content filter criterion for selecting independently generated observation data records for further analysis as containing an identifier selected from a plurality of available identifiers eligible for further analysis.

19. The computer program product of claim 15, wherein the machine learning based model is a linear regression model.

20. The computer program product of claim 15, wherein the sequentially executed plurality of subprocesses are configured to:
exclude one or more observation data records identified as failing to satisfy a rule of a subprocess for an intra-date conflict between cancer stage identifiers within observation data records having a common date; and
exclude one or more observation data records identified as failing to satisfy a rule of a subprocess for an inter-date conflict between cancer stage identifiers within observation data records having different dates.
